SEQUENCE LISTING (17 sequences)

SEQ ID NO: 1 - DNA, 1458 bp, Saccharomyces cerevisiae
atgtctggccaaaagggcgagatcgtggtttatacaaaagagttagaaactacccccgagcttttaccaaaccatgaagttttcaagattaggattggccaaaagctatttgagatctcaggtgctacattgaattcggatgcgcccaattttttcacccaattcttcaatacgcacgataaaaatacgattctatttgttgatagatcggaagacgtgttcatcataatatacagacatttacaaggttattttcccgatattaagaacgaagtacaatttacctgtcttttcgcggacgcattatattttcagttacccaagttagttaaactcataaaagaatacgactaccacttcactaatattggaggtgttccctttaaggtaccaaaatcattattccatgaagaagggaaccggttgaactattttgaaacgatttctcgcatatcttatgaggaaattgagaagtgggagtcaaataagaagccgggctttccacctttattaccaccctcatatattgcaaggtccccagaatttttccgagatatattatctttactgggcggttccaaattagaactgtctgaggaaagaaccgcatctctgataaaagagtgtcgttattaccgtctcaacaggttggagcaggaattagtgcgggcaaaaattatatataatcccttaactaattgtcaagaagtttgcatagctcttgatagcgtctccaagaagggtgtaaccattgaaaggttaacatcacttcataccggaaaccaatctttagcggtaagcagttgtctcaatggaactgagggtgaaaaagccgctactggatttcataaaaccgagactgattcaggcaacaatgatgaatatgaacctccaacaaaaaaggtaaaacattgtattgaaagacactggagcatgttaaaataccaaaggccttatatcgatacagtttcgcacgatcttattttccaattacactctaaccagtgcaaaataatattcaacaaaaagaataagactgttcatgtagacctatccagagaggctgcaatgttgtttgaaaacaagttctcggatgttttattggaaaacccagattttaaaattgatctgtccgaatataaggtcaaacttcgtgacagtcaaatgcaagttgaatcccacttaattattcctgcatgtgtctccatttgtgacttgacggtaaatggcgcaaaatgctgtaatattttctcattagttaacgactccaaatgtaaagggagggtcttggattgcaccaatttgaaagttttgaattgcgttcatggtttgaaactacacttatcgaaatccatgtggaagctaggtacaaataatggaagaatcattctcgttgctgtaaaagccgaaaccttctctgggacgaaggaatattgcaaaatgattgattttctgtga

SEQ ID NO: 2 - protein, 485 aa, Saccharomyces cerevisiae
MSGQKGEIVVYTKELETTPELLPNHEVFKIRIGQKLFEISGATLNSDAPNFFTQFFNTHDKNTILFVDRSEDVFIIIYRHLQGYFPDIKNEVQFTCLFADALYFQLPKLVKLIKEYDYHFTNIGGVPFKVPKSLFHEEGNRLNYFETISRISYEEIEKWESNKKPGFPPLLPPSYIARSPEFFRDILSLLGGSKLELSEERTASLIKECRYYRLNRLEQELVRAKIIYNPLTNCQEVCIALDSVSKKGVTIERLTSLHTGNQSLAVSSCLNGTEGEKAATGFHKTETDSGNNDEYEPPTKKVKHCIERHWSMLKYQRPYIDTVSHDLIFQLHSNQCKIIFNKKNKTVHVDLSREAAMLFENKFSDVLLENPDFKIDLSEYKVKLRDSQMQVESHLIIPACVSICDLTVNGAKCCNIFSLVNDSKCKGRVLDCTNLKVLNCVHGLKLHLSKSMWKLGTNNGRIILVAVKAETFSGTKEYCKMIDFL

SEQ ID NO: 3 - DNA, 1458 bp, Saccharomyces cerevisiae
atgtctggccaaaagggcgagatcgtggtttatacaaaagagttagaaactacccccgagcttttaccaaaccatgaagttttcaatattaggattggccaaaagctatttgagatctcaggtgcttcattgaattcggatgcgcccaattttttcacccaattcttcaatacgcacgataaaaatacgattctatttgttgatagatcggaagacgtgttcatcataatatacagacatttacaaggttattttcccgatattaaggacgaagtacaatttacctgtcttttcgcggatgcattatattttcagttacccaagttagttaaactcataaaagaatacgactaccacttcactaatattggaggtgttccctttaaggtaccaaaatcattattccatgaagaagggaaccggttgaactattttgaaacgatttctcgcacatcttatgaggaaattgagaagtgggagtcaaataagaagccgggctttccacctttattaccaccctcatatattgcaaggtccccagaatttttccgagaaatattatctttactgggcggttctgaattagaactgtctgaggaaagaaccgcatctctgataaaagagtgtcgttattaccgtctcaacaggttggagcaggaattagtgcgggcaaaaattatatataatcccttaactaattgtcaagaagtttgcatagctctcgatagcgtctccaagaagggtgtaaccattgaaaggttaacatcacttcatactggaaaccaatctttagcggtaggcagttgtctcaatggaactgagggtgaaaaaaccgctactggatttcataaaaccgagactgattcaggcaacaatgatgaatctgaacctccaacaaaaaaggtaaaacattgtattgaaagacactggagcatgttaaaataccaaaggccttatatcgatacagtttcgcacgatcttattttccaattacactctaaccagtgcaaaataatattcaacaaaaagaataagactgttcatgtagacctatccagagaggctgcaatgttgtttgaaaacaagttctcggatgttttattggaaaacccagattttaaaattgatctgtccgaatataaagtcaaacttcgtgacagtcaaatgcaagttgaatcccacttaattattcctgcatgtgtctccatttgtgacttgacggtaaatggcgcaaaatgctgtaatattttctcattagttaacgactccaaatgtaaagagagggtcttggattgcaccaatttgaaagttttgaattgcgttcatggtttgaaactacacttatcgaaatccatgtggaagctaggtacaaataatggaagaatcattctcgttgctgtaaaagccgaaaccttctctgggacaaaggaatattgcaaaatgattgattttctgtga

SEQ ID NO: 4 - protein, 485 aa, Saccharomyces cerevisiae
MSGQKGEIVVYTKELETTPELLPNHEVFNIRIGQKLFEISGASLNSDAPNFFTQFFNTHDKNTILFVDRSEDVFIIIYRHLQGYFPDIKDEVQFTCLFADALYFQLPKLVKLIKEYDYHFTNIGGVPFKVPKSLFHEEGNRLNYFETISRTSYEEIEKWESNKKPGFPPLLPPSYIARSPEFFREILSLLGGSELELSEERTASLIKECRYYRLNRLEQELVRAKIIYNPLTNCQEVCIALDSVSKKGVTIERLTSLHTGNQSLAVGSCLNGTEGEKTATGFHKTETDSGNNDESEPPTKKVKHCIERHWSMLKYQRPYIDTVSHDLIFQLHSNQCKIIFNKKNKTVHVDLSREAAMLFENKFSDVLLENPDFKIDLSEYKVKLRDSQMQVESHLIIPACVSICDLTVNGAKCCNIFSLVNDSKCKERVLDCTNLKVLNCVHGLKLHLSKSMWKLGTNNGRIILVAVKAETFSGTKEYCKMIDFL

SEQ ID NO: 5 - DNA, 1488 bp, Saccharomyces cerevisiae
atgtcaaattctccaacagtagcaactttgtcacaagaatatttcgatccgaatattcctcaaatactcccacacgagaaaatgtataagattcaggtggggaaatcattattcaaaatcagcggtgcttctttaagctcggatggtcctagtttcttcaccgaatatttttctaagaagcgctctccttccaataacgatgacagtaataacgataccatggaatctaataaaaatgaggttctattcatagacagatcagccgaagtttttgaatggatctatcaacatttgcaaggatatataattgagattaaggatgaagtacaatataccatgcttttcgcagatgcgatgtactacaatttaccgcgattgcgatctttactgaaagaaacggactattattttaccaacattggtggacaatcattcaagatagcgaaaaatttatttagaagagaaggagattccccaaattatttcgaaatatatgcggctacagtttatatcgatgtggaagagctgataatatctaaaaaactattaaggcctccatctcattctgctccttacattccaagatcttctgagtacttcaaagatttgctgacgttactgggaggagcatccatcgatctagatgataataaaagaaatgccttgataaaagagtgccgttattacaggcttttaaatctcgaacagcggctaatcaaatctcatattagctataacccaataacaaggaaagaagagatttgcctcctattgaaggatttgtcaaaaaaaggcatcacttttccagcttcttctgcattttcaacctcgccctactttgaagacgatttctgtagcataaatgaatgcgactcgctatcaaaaacccgtgaacagccagcaaataaaaagattaaacttgacatgactgaaaaatataacgattcctggaatatgctttgttataagagaccttttttagataagcatccgagagagttgatatttcaaataaattcgactgattgtacgataatcctcaataaggaaagccaaagtatccatgtagacataactggagaatccgcctataagtttgaggccttatttggaagtcatttgccaaatacaccctctggtgcgcctaagttgaaaaattaccagtatcggttcccatccgatagtacgcagacgaaaattgaaactcattatttacttccagcatgcatatatctctgtgatttggacataaacggcattaagatttctcaagttcaaactttgctaactgataaaaataaattcaatgacagggtcattgacgtgtctgatccacttgaccttagattttgttctggtttaaagctttacttaagaaagtctctctggaaactggccgtgaaagatggcaatattatgctgatagctattaaagctatagcatttaacggaactaaggaatactacaagggatatgaatatctgtag

SEQ ID NO: 6 - protein, 495 aa, Saccharomyces cerevisiae
MSNSPTVATLSQEYFDPNIPQILPHEKMYKIQVGKSLFKISGASLSSDGPSFFTEYFSKKRSPSNNDDSNNDTMESNKNEVLFIDRSAEVFEWIYQHLQGYIIEIKDEVQYTMLFADAMYYNLPRLRSLLKETDYYFTNIGGQSFKIAKNLFRREGDSPNYFEIYAATVYIDVEELIISKKLLRPPSHSAPYIPRSSEYFKDLLTLLGGASIDLDDNKRNALIKECRYYRLLNLEQRLIKSHISYNPITRKEEICLLLKDLSKKGITFPASSAFSTSPYFEDDFCSINECDSLSKTREQPANKKIKLDMTEKYNDSWNMLCYKRPFLDKHPRELIFQINSTDCTIILNKESQSIHVDITGESAYKFEALFGSHLPNTPSGAPKLKNYQYRFPSDSTQTKIETHYLLPACIYLCDLDINGIKISQVQTLLTDKNKFNDRVIDVSDPLDLRFCSGLKLYLRKSLWKLAVKDGNIMLIAIKAIAFNGTKEYYKGYEYL

SEQ ID NO: 7 - protein, 215 aa, Saccharomyces cerevisiae
MLGIRSSVKTCFKPMSLTSKRLISQSLVASKSTYRTPNFDDVLKENNDADKGRSYAYFMVGAMGLLSSAGAKSTVETFISSMTATADVLAMAKVEVNLAAIPLGKNVVVKWQGKPVFIRHRTPHEIQEANSVDMSALKDPQTDADRVKDPQWLIMLGICTHLGCVPIGEAGDFGGWFCPCHGSHYDISGRIRKGPAPLNLEIPAYEFDGDKVIVG

SEQ ID NO: 8 - protein, 92 aa, Saccharomyces cerevisiae
MLGIRSSVKTCFKPMSLTSKRLISQSLVASKSTYRTPNFDDVLKENNDADKGRSYAYFMVGAMGLLSSAGAKSTVETFISSMTATADVLAMA

SEQ ID NO: 9 - protein, 125 aa, Saccharomyces cerevisiae
MAKVEVNLAAIPLGKNVVVKWQGKPVFIRHRTPHEIQEANSVDMSALKDPQTDADRVKDPQWLIMLGICTHLGCVPIGEAGDFGGWFCPCHGSHYDISGRIRKGPAPLNLEIPAYEFDGDKVIVG

SEQ ID NO: 10 - DNA, 1665 bp, Saccharomyces cerevisiae
atgaccgcaaagacttttctactacaggcctccgctagtcgccctcgtagtaaccattttaaaaatgagcataataatattccattggcgcctgtaccgatcgccccaaataccaaccatcataacaatagttcgctggaattcgaaaacgatggcagtaaaaagaagaagaagtctagcttggtggttagaacttcaaaacattgggttttgcccccaagaccaagacctggtagaagatcatcttctcacaacactctacctgccaacaacaccaataatattttaaatgttggccctaacagcaggaacagtagtaataataataataataataacatcatttcgaataggaaacaagcttccaaagaaaagaggaaaataccaagacatatccagacaatcgatgaaaagctaataaacgactcgaattacctcgcatttttgaagttcgatgacttggaaaatgaaaagtttcattcttctgcctcctccatttcatctccatcttattcatctccatctttttcaagttatagaaatagaaaaaaatcagaattcatggacgatgaaagctgcaccgatgtggaaaccattgctgctcacaacagtctgctaacaaaaaaccatcatatagattcttcttcaaatgttcacgcaccacccacgaaaaaatcaaagttgaacgactttgatttattgtccttatcttccacatcttcatcggccactccggtcccacagttgacaaaagatttgaacatgaacctaaattttcataagatccctcataaggcttcattccctgattctccagcagatttctctccagcagattcagtctcgttgattagaaaccactccttgcctactaatttgcaagttaaggacaaaattgaggatttgaacgagattaaattctttaacgatttcgagaaacttgagtttttcaataagtatgccaaagtcaacacgaataacgacgttaacgaaaataatgatctctggaattcttacttacagtctatggacgatacaacaggtaagaacagtggcaattaccaacaagtggacaatgacgataatatgtctttattgaatctgccaattttggaggaaaccgtatcttcagggcaagatgataaggttgagccagatgaagaagacatttggaattatttaccaagttcaagttcacaacaagaagattcatcacgtgctttgaaaaaaaatactaattctgagaaggcgaacatccaagcaaagaacgatgaaacctatctgtttcttcaggatcaggatgaaagcgctgattcgcatcaccatgacgagttaggttcagaaatcactttggctgacaataagttttcttatttgcccccaactctagaagagttgatggaagagcaggactgtaacaatggcagatcttttaaaaatttcatgttttccaacgataccggtattgacggtagtgccggtactgatgacgactacaccaaagttctgaaatccaaaaaaatttctacgtcgaagtcgaacgctaacctttatgacttaaacgataacaacaatgatgcaactgccaccaatgaacttgatcaaagcagtttcatcgacgaccttgacgaagatgtcgattttttaaaggtacaagtattttga

SEQ ID NO: 11 - protein, 554 aa, Saccharomyces cerevisiae
MTAKTFLLQASASRPRSNHFKNEHNNIPLAPVPIAPNTNHHNNSSLEFENDGSKKKKKSSLVVRTSKHWVLPPRPRPGRRSSSHNTLPANNTNNILNVGPNSRNSSNNNNNNNIISNRKQASKEKRKIPRHIQTIDEKLINDSNYLAFLKFDDLENEKFHSSASSISSPSYSSPSFSSYRNRKKSEFMDDESCTDVETIAAHNSLLTKNHHIDSSSNVHAPPTKKSKLNDFDLLSLSSTSSSATPVPQLTKDLNMNLNFHKIPHKASFPDSPADFSPADSVSLIRNHSLPTNLQVKDKIEDLNEIKFFNDFEKLEFFNKYAKVNTNNDVNENNDLWNSYLQSMDDTTGKNSGNYQQVDNDDNMSLLNLPILEETVSSGQDDKVEPDEEDIWNYLPSSSSQQEDSSRALKKNTNSEKANIQAKNDETYLFLQDQDESADSHHHDELGSEITLADNKFSYLPPTLEELMEEQDCNNGRSFKNFMFSNDTGIDGSAGTDDDYTKVLKSKKISTSKSNANLYDLNDNNNDATATNELDQSSFIDDLDEDVDFLKVQVF

SEQ ID NO: 12 - protein, 77 aa, Saccharomyces cerevisiae
MAYTSHLSSKTGLHFGRLSLRSLTAYAPNLMLWGGASMLGLFVFTEGWPKFQDTLYKKIPLLGPTLEDHTPPEDKPN

SEQ ID NO: 13 - DNA, 20 bp, Artificial Sequence
  guide RNA
gttaacgactccaaatgtaa

SEQ ID NO: 14 - DNA, 20 bp, Artificial Sequence
  guide RNA
taatgcgtccgcgaaaagac

SEQ ID NO: 15 - DNA, 20 bp, Artificial Sequence
  guide RNA
ggttttatgaaatccagtag

SEQ ID NO: 16 - DNA, 20 bp, Artificial Sequence
  guide RNA
ttcatggtttggtaaaagct

SEQ ID NO: 17 - DNA, 20 bp, Artificial Sequence
  guide RNA
atattccttcgtcccagaga

(19) United States
(12) Patent Application Publication
(10) Pub. No.: US 2022/0119460 A1
(43) Pub. Date: Apr. 21, 2022

(54) Means and Methods to Overrule Glucose-Mediated Repression of Respiration in Yeast

(71) Applicants: VIB VZW, Gent (BE); KATHOLIEKE UNIVERSITEIT LEUVEN, K.U.LEUVEN R&D, Leuven (BE)

(72) Inventors: Kevin Verstrepen, Leuven (BE); Lieselotte Vermeersch, Balen (BE); Abbas Jariani, Tehran (IR); Bram Cerulus, Wijgmaal (BE); Gemma Perez, Barcelona (ES)

(21) Appl. No.: 17/426,827

(22) PCT Filed: Jan. 29, 2020
(86) PCT No.: PCT/EP2020/052134

§ 371 Date: Jul. 29, 2021

(30) Foreign Application Priority Data
Jan. 30, 2019 (GB) 1901262.4

(51) Int. Cl. C07K 14/395; C12N 1/18; C12N 15/113

(52) U.S. Cl. CPC C07K 14/395; C12N 2310/20; C12N 15/113; C12N 1/18

(57) ABSTRACT

The present invention relates to the field of biochemistry, particularly to the field of yeast fermentations and yeast biomass production, more particularly to modulation of oxidative respiration in yeast. In this application it is disclosed that increasing the respiratory activity in yeast cells reduces the lag phase when switching said yeast cells from glucose to other nutrients. Additionally, a new mutant yeast allele was found that significantly reduces the lag time. The means and methods described herein solve the problem of microbial growth arrest during industrial fermentations when yeasts have to switch to other nutrient sources and provide solutions for the troublesome yeast biomass production.

### CROSS-REFERENCE TO RELATED APPLICATIONS

This application is a national phase entry under 35 U.S.C. § 371 of International Patent Application PCT/EP2020/052134, filed Jan. 29, 2020, designating the United States of America and published in English as International Patent Publication WO 2020/157112 on Aug. 6, 2020, which claims the benefit under Article 8 of the Patent Cooperation Treaty to United Kingdom Patent Application Serial No. 1901262.4, filed Jan. 30, 2019, the entireties of which are hereby incorporated by reference.

### FIELD OF THE INVENTION

The present invention relates to the field of biochemistry, particularly to the field of yeast fermentations and yeast biomass production, more particularly to modulation of oxidative respiration in yeast. In this application it is disclosed that increasing the respiratory activity in yeast cells reduces the lag phase when switching said yeast cells from glucose to other nutrients. Additionally, a new mutant yeast allele was found that significantly reduces the lag time. The means and methods described herein solve the problem of microbial growth arrest during industrial fermentations when yeasts have to switch to other nutrient sources and provide solutions for the troublesome yeast biomass production.

### BACKGROUND

Cells constantly adapt to environmental fluctuations. When faced with environmental changes, microbes often enter a temporary growth arrest during which they reprogram the expression of specific genes to adapt to the new conditions. This phase is called the lag phase. A prime example of such a lag phase occurs when microbes need to switch from glucose to other, less-preferred carbon sources. The lag phase is unquestionably one of the prime characteristics of microbial growth. A short lag time allows cells to resume growth more quickly than competitors with a longer lag. Moreover, the lag time is of crucial importance in industrial fermentations where microbes are often faced with a complex mixture of sugars in challenging environments. These complex media often result in long lag phases during which the fermentation speed is highly reduced. Such so-called “stuck” or “sluggish” fermentations cause great economic losses because they reduce the efficiency of a production plant and might lead to inferior end products that contain undesirable fermentable sugars which make the product sweet and vulnerable to spoilage. It is thus advantageous to develop means and methods to reduce lag times during industrial yeast fermentations.

Despite the immense importance of the lag phase, surprisingly little is known about the exact molecular processes that determine its duration. It is however well established that glucose represses genes involved in the uptake and metabolism of alternative carbon sources. This process is called glucose repression (Diaz-Hernandez et al 2010 Front Physiol 1:22; Conrad et al 2014 FEMS Microbiol Rev 38:254-299). Apart from repressing the metabolism of alternative carbon sources, glucose also affects the expression of genes related to other cellular functions such as respiration, gluconeogenesis, and general stress response mechanisms. The repression of respiration in Saccharomyces yeasts growing in glucose-containing environments explains why S. cerevisiae prefers a fermentative dissimilation of glucose over respiration even in the presence of excess oxygen (Verstrepen et al 2004 Trends biotechnol 22:531-537; Geladé et al 2003 Genome Biol 4:233; de Deken 1966 J Gen Microbiol 44:149-156). Said Crabtree-effect is particularly troublesome in the field of yeast biomass production, where alcoholic fermentation is nowadays prevented by intensive aeration, mixing and carefully dosing glucose. In the field of beer and wine production, the almost spontaneous production of alcohol upon glucose fermentation makes it extremely difficult to efficiently produce alcohol-free beer or wine.

Current application discloses that the transition from fermentative to respirative metabolism is a key bottleneck for cells to overcome the lag phase. Using the molecular toolbox of S. cerevisiae combined with detailed growth experiments, the key molecular processes and genes that influence lag duration are revealed. Hence, these findings provide solutions to a plethora of problems encountered during yeast-related industrial activities and can lead to an increased efficiency of industrial fermentations, to simplify yeast biomass production or to avoiding or reducing the alcohol-producing fermentation events during beer and wine production.

### SUMMARY

In a first part of the application it is demonstrated that oxidative respiration in yeast is linked to the duration of the lag phase associated with depletion of glucose in the growth medium of said yeasts. Methods are provided to reduce the lag phase of a culture of yeast cells, comprising the step of stimulating respiration in said yeast cells. Said stimulation can be performed by adding an agonist of yeast respiration to the growth medium, by enhancing the expression of an activator of respiration in said yeast cells and/or by reducing the expression of a repressor of respiration in said yeast cells. In particular embodiments, said activator of respiration is the Hap4 transcription factor or the N-Rip1 protein. In other particular embodiments, said repressor of respiration is YLR108C or YDR132C. The application also discloses that complexes III and IV of the electron transport chain are important for the methods described above. Means and methods are therefore provided to activate or stabilize said complexes in yeast.

In a second part of the application a novel repressor of oxidative respiration in yeast is disclosed. Methods are provided to increase oxidative respiration in yeast comprising reducing the expression of YLR108C or YDR132 in said yeast cells. Given that increased respiration shortens the lag phase upon glucose depletion, reducing the expression of YLR108C or YDR132 in yeast is also provided to reduce the lag phase of a culture of said yeasts. In a particular embodiment, the application provides a chimeric gene construct comprising a promoter active in yeast operably linked to a Crispr guide RNA targeting a YLR108C or YDR132C allele. The application further provides the uses of said chimeric gene construct to increase respiration in a yeast culture, to reduce the lag phase of a culture of said yeast cells, to enhance biomass production of said yeast cells or to produce beer or wine with reduced alcohol levels.

Description of the Figures The patent or application file contains at least one drawing executed in color. Copies of this patent or patent application publication with color drawing(s) will be provided by the Office upon request and payment of the necessary fee.

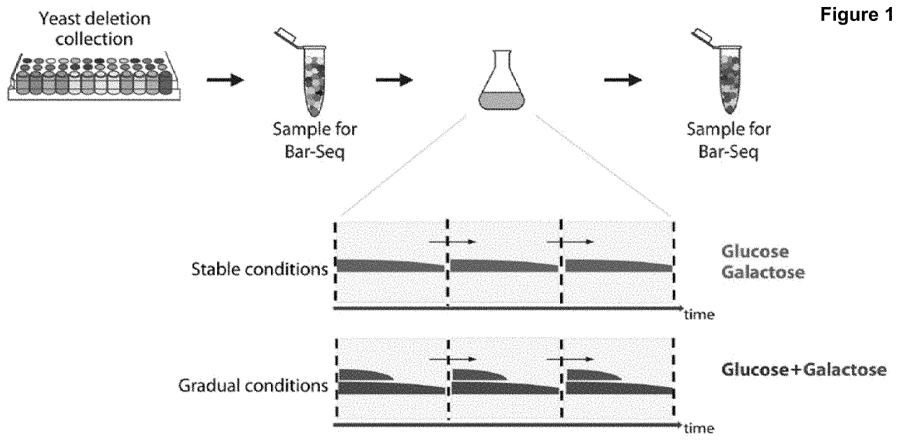

FIG. 1 is a schematic representation of the genome-wide Bar-Seq screen set-up. All the mutants from the yeast deletion collection were pooled in an aliquot and grown in two different regimes (stable and gradual conditions). For selection in stable conditions, the mutants were grown in media containing a single carbon source. For selection in gradual conditions, the pool of mutants was grown in low glucose (0.5%) supplemented with galactose (5%). At the start and after 3 rounds of selection, an initial sample and a final sample, respectively, were taken. These samples were used to determine enrichment of mutants through Bar-Seq.

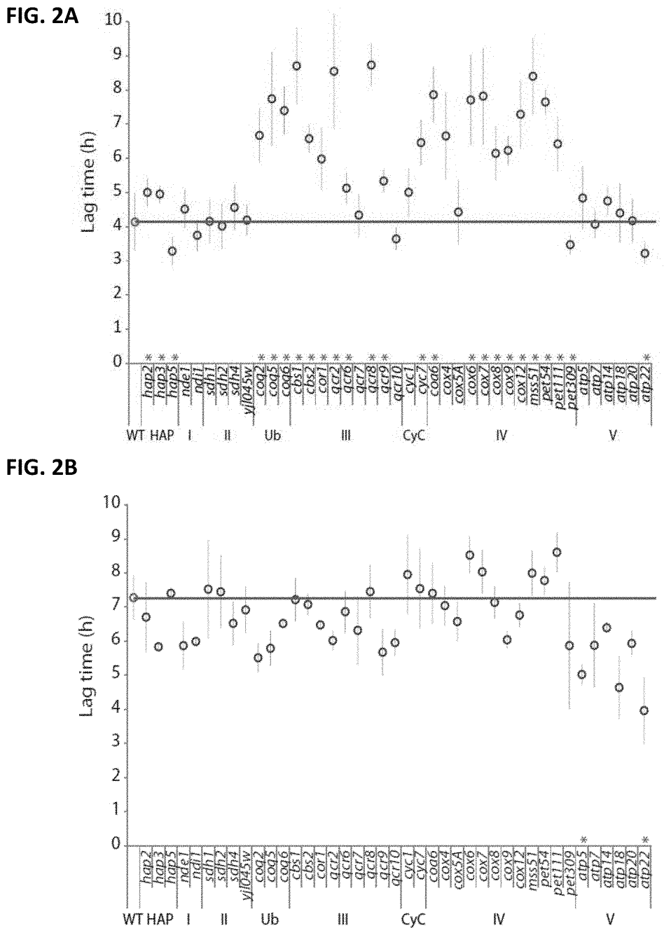

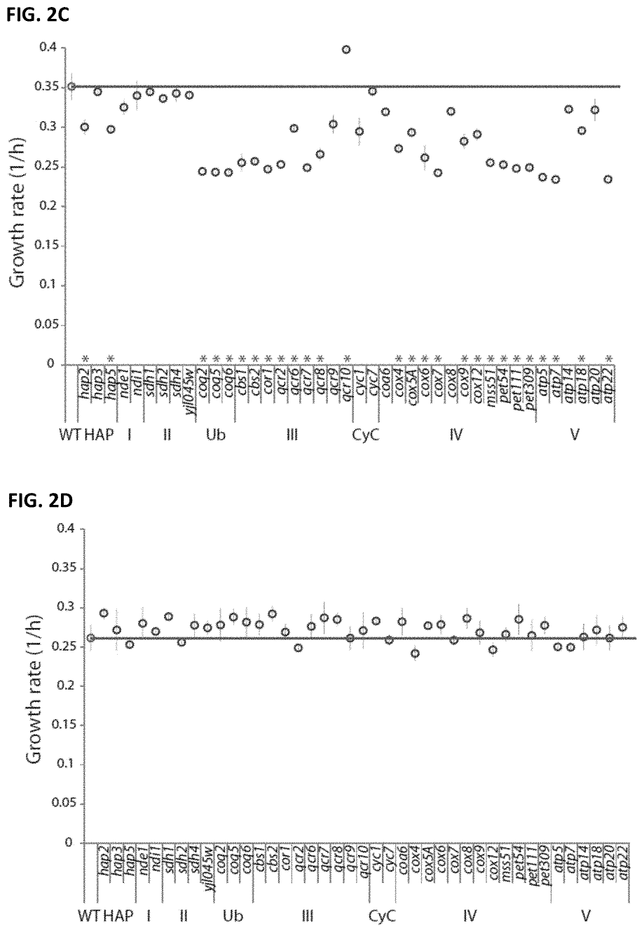

FIGS. 2A-2D show that the respiratory activity affects the duration of the glucose-to-galactose lag phase. FIG. 2A and FIG. 2B show the population lag times of the 41 respiratory deletion mutants growing on low glucose supplemented with galactose with (FIG. 2B) or without (FIG. 2A) addition of antimycin A to the media. The red circle and line indicate the lag time of the wild type. Error bars correspond to standard deviations of 3 or more biological replicates. *, P<0.05 (two-tailed Student's t test).

FIG. 2C AND FIG. 2D show the growth rate of the 41 respiratory deletion mutants growing in stable sugar conditions (5% galactose) with (FIG. 2D) or without (FIG. 2C) addition of antimycin A to the media. Error bars correspond to standard deviations of 3 or more biological replicates. *, P<0.05 (two-tailed Student's t test).

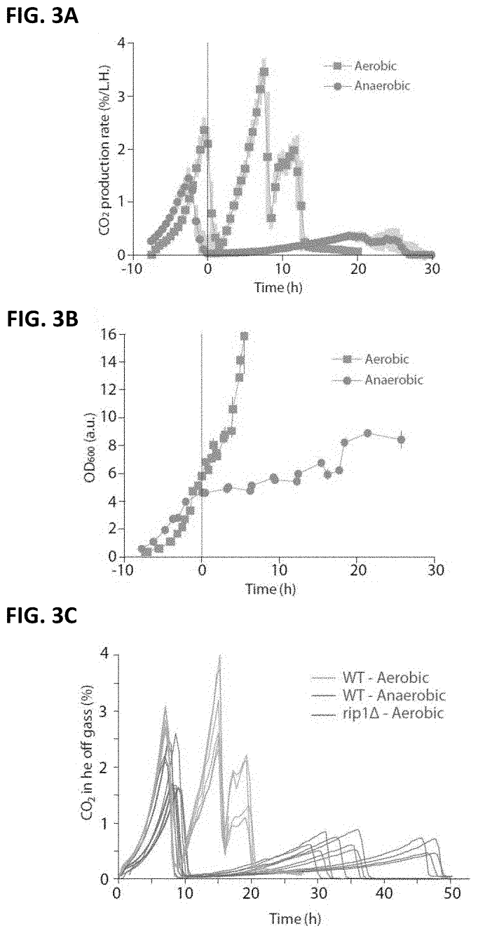

FIGS. 3A-3C illustrate that respiration affects lag phase independent of the background.

FIG. 3A shows the volumetric CO2 production rate profiles of bioreactor batch cultures of S. cerevisiae CEN.PK113-7D grown on glucose-galactose mixtures in aerobic (orange rectangles) and anaerobic (blue circles) conditions. The shadows around the curves correspond to the standard deviation of 6 (aerobic) and 7 (anaerobic) biological replicates.

FIG. 3B shows the optical density over time for aerobic and anaerobic growth of S. cerevisiae CEN.PK113-7D. Error bars correspond to standard deviations of 2 biological replicates. Optical densities corresponded well with cell weight (dry weight) concentrations.

FIG. 3C shows that anaerobic conditions or deletion of a complex III subunit (RIP1) leads to a prolonged lag phase in CEN.PK113-7D.

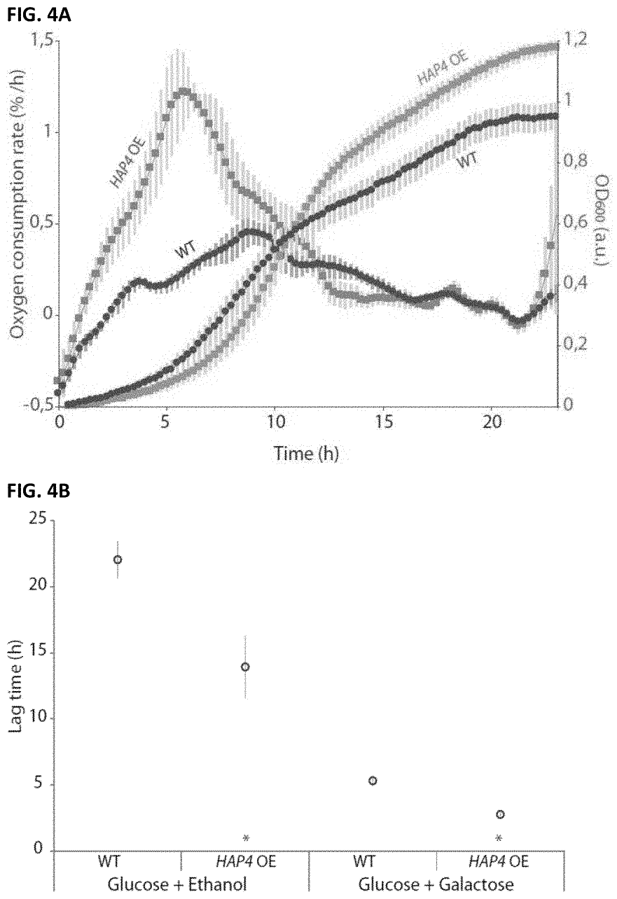

FIGS. 4A and 4B. demonstrate that HAP4 overexpression reduces the lag phase.

FIG. 4A shows the oxygen consumption rate (light/dark blue) and growth curve (orange/red) for the wild type (circles) and a strain with HAP4 overexpression (rectangles). Error bars correspond to standard deviations of 5 biological replicates.

FIG. 4B shows the population lag time of wild type and the HAP4 OE strain when shifting from glucose to either ethanol or galactose. Error bars correspond to standard deviations of 3 or more biological replicates. *, P<0.05 (two-tailed Student's t test).

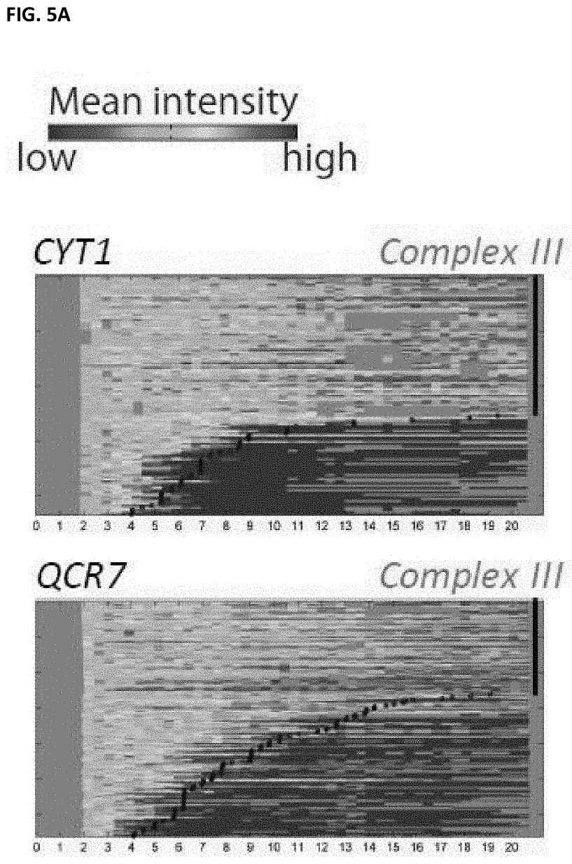

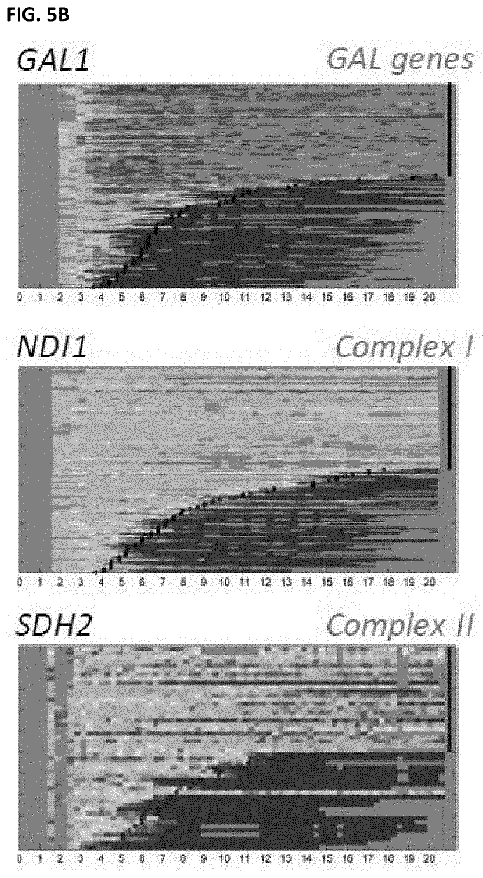

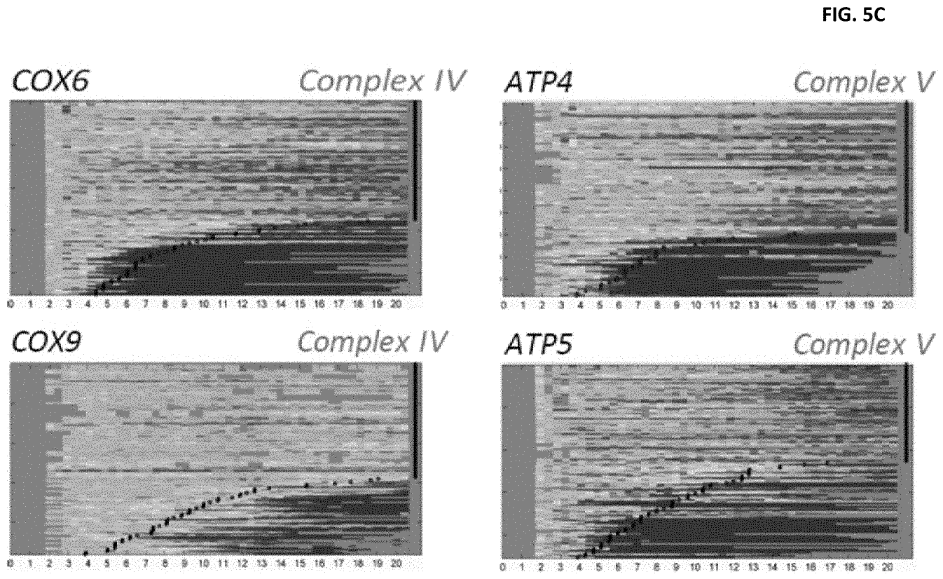

FIGS. 5A-5C. illustrate that genes involved in galactose metabolism and respiration are induced prior to the cells' escape from the lag phase. The figure shows so-called kymographs of single cells during growth on galactose after 8 h of growth on glucose. Microcolony growth of individual single cells expressing a specific fluorescently tagged protein was tracked (the corresponding gene name is specified in the panel). Every row represents the mean fluorescent intensity profile of the microcolony grown from a single cell. Black squares depict the moment at which each single cell starts growing on galactose. Note that the GAL1 gene as well as genes involved in respiration (NDI1, CYT1, COX6, ATP4, SDH2, QCR7, COX9, and ATP5) are induced prior to each cell's escape from the lag phase and that a large fraction of cells (typically 40% to 60% of the population) fail to induce these genes and also fail to resume growth on galactose.

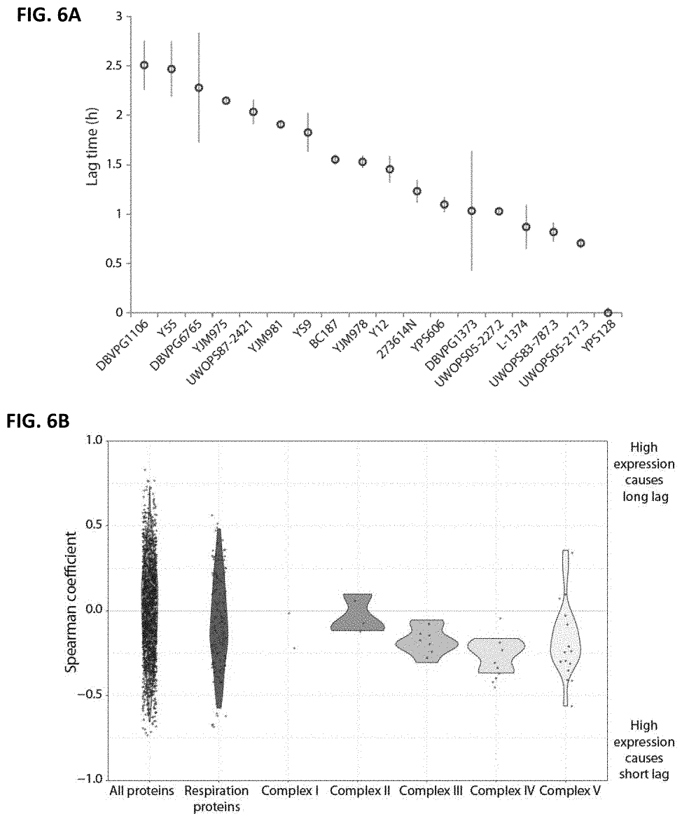

FIGS. 6A and 6B. demonstrate that the glucose-to-galactose lag times of different natural S. cerevisiae strains correlate with their expression of respiratory proteins in medium containing glucose.

FIG. 6A shows the population lag times of 18 different S. cerevisiae strains during a gradual shift from glucose to galactose. Error bars correspond to standard deviations of 3 or more biological replicates.

FIG. 6B shows violin plots of the Spearman correlation coefficients between the protein expression obtained by Skelly et al. (2013) of 18 S. cerevisiae natural strains and their lag times when shifting from glucose to galactose. Red dots indicate the correlation coefficient of individual proteins.

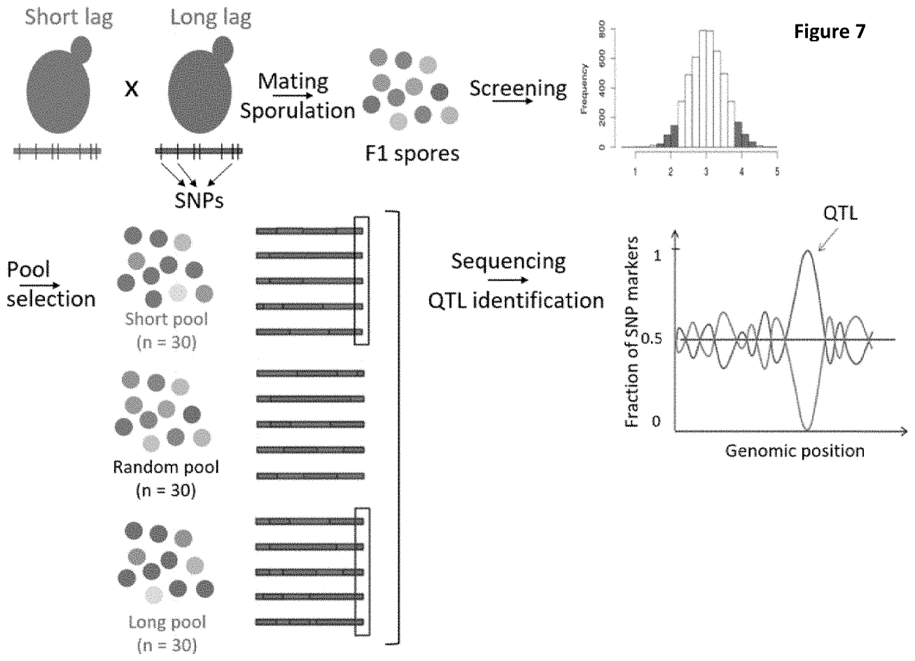

FIG. 7 is a schematic representation of the bulk segregant QTL analysis. A short lag and long lag phase strain were crossed and subsequently sporulated to obtain a F1 population that was screened for lag phase length for a gradual shift from glucose to ethanol. From said screen a population with short lag time (short pool) was selected as well as a population with long lag time (long pool), and additionally a control population (random pool). The long pool is not visualised. Finally, DNA was extracted from the parental strains and the segregating pools for QTL mapping.

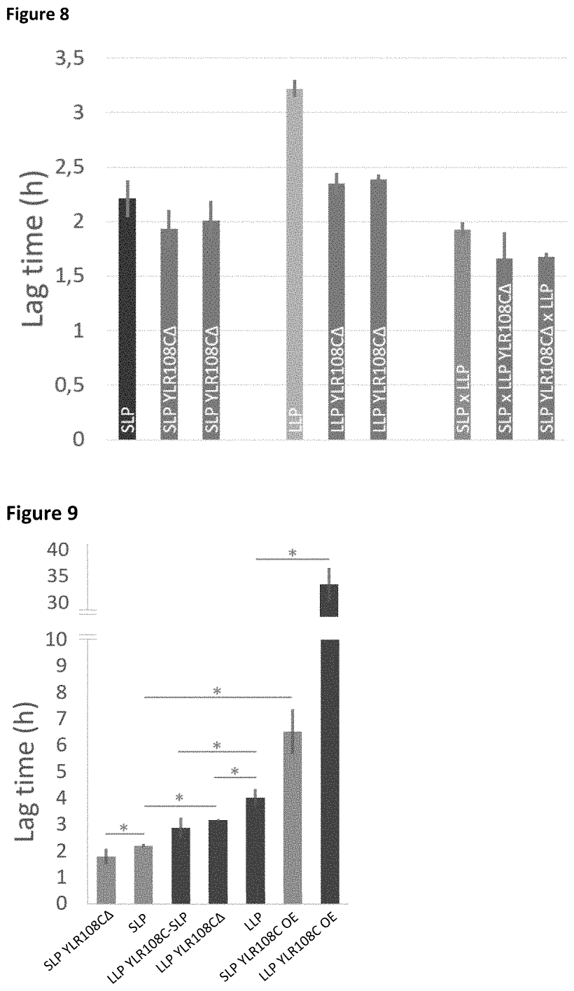

FIG. 8 shows the lag times in hours (from left to right) for the short-lag phase strain (1^{st} bar), the short-lag phase strain wherein the YLR108C allele was deleted (2^{nd} and 3^{rd} bar), the long-lag phase strain (4^{th} bar), the long-lag phase strain wherein the YLR108C allele was deleted (5^{th} and 6^{th} bar), the cross between short-lag and long-lag phase strain (7^{th} bar), for said cross wherein the long-lag phase YLR108C allele was deleted (8^{th} bar) and for said cross wherein the short long-lag phase YLR108C allele was deleted (9^{th} bar).

FIG. 9 shows the lag times in hours (from left to right) for the short-lag phase strain (SLP) wherein the YLR108C allele was deleted (SLP YLR108CA), the SLP, the long-lag phase strain wherein the long-lag YLR108C allele was replaced with the short-lag YLR108C allele (LLP YLR108C-SLP), the long-lag phase strain wherein the long-lag YLR108C allele was deleted (LLP YLR108CA), the long-lag phase strain (LLP), the SLP wherein the short-lag YLR108C was overexpressed (SLP YLR108C OE) and the LLP strain wherein the long-lag YLR108C allele was overexpressed (LLP YLR108C-OE).

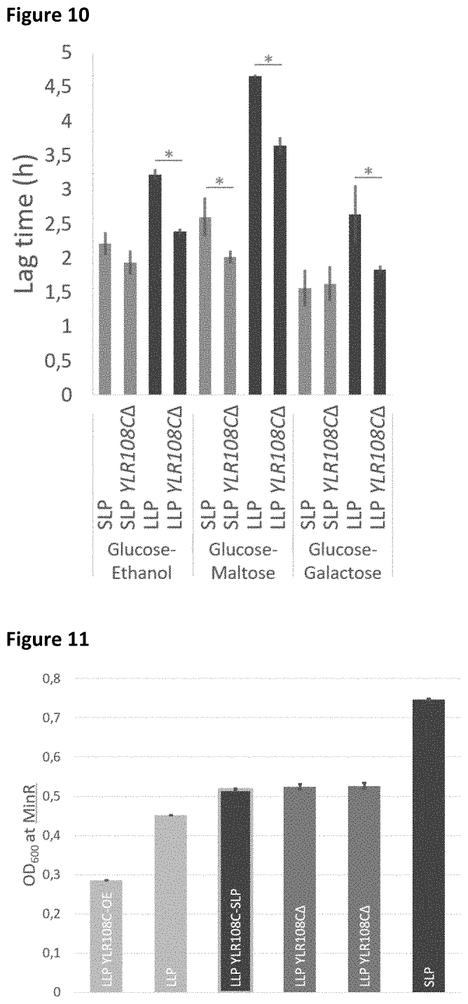

FIG. 10 illustrates that the lag time of the long-lag phase strain (LLP) is reduced when its YLR108C allele is deleted (LLP YLR108CA) both in environments where the nutrient source changes from glucose to ethanol (left), from glucose to maltose (middle) or from glucose to galactose (right). Deleting the YLR108C allele in the short-lag phase (SLP) did not further reduce the lag time for glucose-ethanol or glucose-galactose changes, but had an effect for glucose-maltose changes.

FIG. 11 shows the OD₆₀₀ at MinR (i.e. a measure for the population density at which the cells enter the lag phase) from a series of yeast cultures of the short-lag phase yeast (SLP), the long-lag phase yeast (LLP) or long-lag phase yeast wherein the YLR108C was overexpressed (LLP YLR108C-OE), wherein the YLR108C was replaced by the short-lag phase YLR108C allele (LLP YLR108C-SLP) or wherein the YLR108C allele was deleted (LLP YLR108CA). All yeast strains were grown in changing nutrient conditions from glucose-to-ethanol.

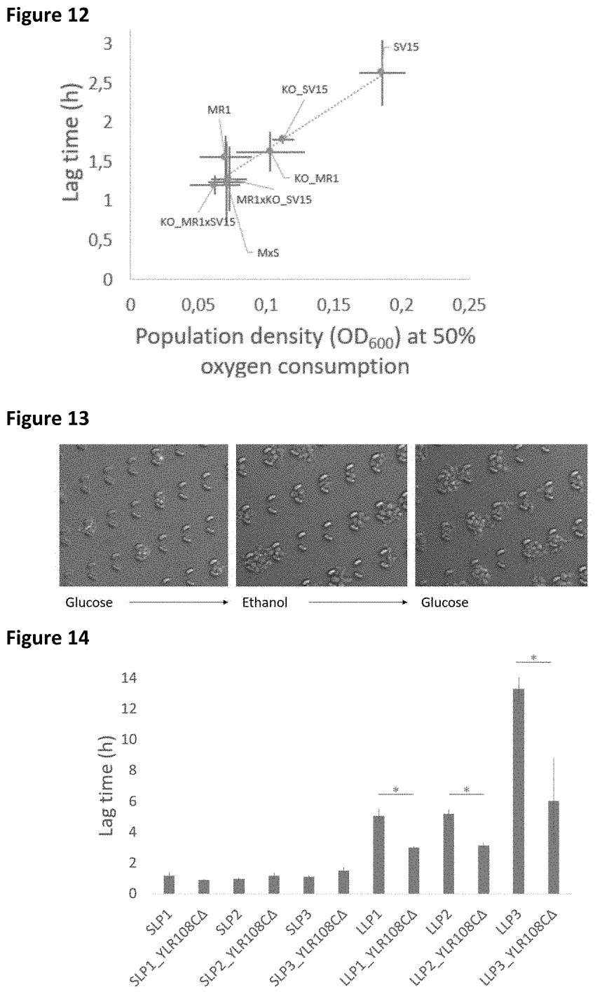

FIG. 12 shows the correlation between the lag time of yeast cultures and their population density at 50% oxygen consumption (R² is 0.9492). Cultures with a low density at 50% oxygen consumption (such as the short lag phase strain (MR1) and the long-lag phase strain wherein the YLR108C allele has been deleted (KO_SV15) have a high respiratory activity and consequently have short lag times compared to cultures with a high density at 50% oxygen consumption (such as the long-lag phase strain (SV15)).

FIG. 13 shows an overlay of a bright field and a fluorescence picture of yeast cells producing a fluorescent YLR108C (YLR108C-Venus fusion). Expression of YLR108C is induced in the presence of glucose (right picture), repressed when nutrients are changed from glucose to ethanol (middle picture) and re-induced when adding glucose to the medium.

FIG. 14 illustrates that reducing the expression of the endogenous YLR108C allele reduces the lag time (in hours) for different long-lag strains (LLP1-LLP2-LLP3).

### DETAILED DESCRIPTION

Despite its industrial relevance, the genetic network that determines the duration of the lag phase has not been studied in much detail. The inventors of current application performed a series of experiments aiming at assessing which genes determine the duration of the lag phase when yeast cells switch from glucose to less-preferred carbon sources. First, the Bar-Seq technology was used as described in Shoemaker et al. (1996, Nat Genet 14:450-456) in combination with a genome-wide S. cerevisiae deletion collection of 4,887 single-deletion mutants as described in Winzeler et al. (1999 Science 285:901-906). Briefly, Bar-Seq takes advantage of the DNA barcode carried by each mutant in said collection. Specifically, each deletion mutant has two different unique 20-bp barcode sequences, the so-called up (UP) and down (DN) barcodes, each flanked by primer sequences that are common across all mutants. These DNA tags make it possible to pool all 4,887 deletion mutants into one mixed culture and, by counting the proportion of the different barcodes using high-coverage sequencing, measure changes in the relative proportion of each of the 4,887 mutants in this pooled culture. Hence, when the pooled population of mutants is subjected to environmental changes, the relative fitness of each mutant can be determined by comparing the change in relative frequency between the initial and final samples of each experiment.

The inventors of current application set out to investigate the duration of the lag phase of the 4,887 available single-gene deletion mutants during a gradual switch from glucose to galactose. To obtain strong enrichment and depletion of mutants, the population was subjected to three rounds of this selection regime (FIG. 1). Additional adaptations were made to specifically assess the effect of the gene deletion on the lag phase duration and to minimize the effect of gene deletion on the growth speed in either glucose or galactose (for more details see Perez-Samper et al 2018 mBio 9: e01331-18). Following the Bar-Seq experiments, an enrichment score for each individual mutant under each condition was calculated and subsequently corrected for the deletions that cause general slow growth. Based on this score, 200 deletion mutants whose knockout genes resulted in longer lag phases were chosen for interaction network analysis. The interaction network analysis is based on known physical, genetic, and regulatory interactions and allows visualizing how the different genes that were picked up in the screen are connected by a common biological process or pathway (De Maeyer et al 2013 Mol Biosyst 9:1594-1603). When comparing the interaction network corresponding to the genes with the strongest depletion during a glucose-to-galactose shift and the corresponding network during growth in stable galactose, it could be confirmed that the GAL genes which are directly involved in the uptake and metabolism of galactose (Rohde et al 2000 Mol Cell Biol 20:3880-3886) are crucial for escaping the lag phase. However, it was surprisingly found that mitochondrial genes are also a common shared group in these two networks. This suggests that the expression of respiratory genes plays a role both in growth on galactose and in the metabolic adaptation during a glucose-to-galactose shift. This is an unexpected and interesting result, since both glucose and galactose are known to sustain growth using fermentation, as opposed to glycerol or ethanol, which require respiration.

The experiments described in the Examples further revealed that genes involved in respiration, and specifically those encoding complexes III and IV of the electron transport chain, are needed for efficient growth resumption after the lag phase. Aerobic growth experiments in presence of antimycin A (a respiration inhibitor) as well as anaerobic growth experiments confirmed the importance of respiratory energy conversion in determining the lag phase duration. Moreover, while mutants in mitochondrial respiratory components have a long lag phase, overexpression of a stimulator of respiration, for example the HAP4 transcription factor, leads to significantly shorter lag phases. Together, these results suggest that the glucose-induced repression of respiration, known as the Crabtree effect, is a major determinant of microbial fitness in fluctuating carbon environments.

Importantly, the Crabtree effect is not restricted to Saccharomyces as it has been shown that several other yeasts show a similar tendency toward fermentation and are Crabtree positive (Hagman et al 2014 FEBS J 281:4805-4814; Hagman et al 2015 PLoS One 10:e0116942). Fully in line with this, the correlation between respiration and lag time disclosed herein is valid independently of the Saccharomyces background.

Based hereon, the invention is defined in the following aspects and embodiments.

In a first aspect, the use of an agonist of yeast respiration is provided to reduce the lag phase of a yeast culture. This is equivalent as saying that a method is provided to reduce the lag phase of a yeast culture, wherein said method provides the step of stimulating the respiration or cellular respiration in said yeast cells. The “lag phase” or “lag time” as used herein refers to the period of time needed for yeast cells growing in a first carbon source to reprogram their metabolism and express the enzymes required for growth in a secondary carbon source. Hence, the use of an agonist of yeast respiration is provided to reduce the lag phase of a yeast culture when said culture switches from a first to a second carbon source.

In one embodiment, said carbon source is a carbohydrate.

A “carbohydrate” as used herein is a biomolecule consisting of carbon (C), hydrogen (H) and oxygen (O) atoms. In one non-limiting example said biomolecule may have a hydrogen-oxygen atom ratio of 2:1 (as in water) and thus has the empirical formula Cₘ(H₂O)ₙ (where m may be different from n). This formula holds for example true for monosaccharides. Indeed, the term “carbohydrate” is most common in biochemistry, where it is a synonym of “saccharide”, a group that comprises sugars, starch, and cellulose. The saccharides are divided into four chemical groups: monosaccharides, disaccharides, oligosaccharides, and polysaccharides. Monosaccharides and disaccharides, the smallest (lower molecular weight) carbohydrates, are commonly referred to as sugars. Non-limiting examples include the monosaccharides fructose and glucose and the disaccharides sucrose and lactose.

In another embodiment a method is provided to reduce the lag phase of a yeast culture, comprising the step of stimulating the respiration in said yeast cells when said culture switches from a first to a second carbon source. In a particular embodiment, said first carbon source is glucose, said second carbon source is any carbohydrate that is not glucose and consequently said lag phase is associated with glucose depletion. Non-limiting examples of such non-glucose second carbon sources are galactose, maltose, glycerol, ethanol, . . . .

Alternatively, in a particular embodiment, the “lag phase” can also refer to the period of time needed for yeast cells to reprogram their metabolism and express the enzymes required for growth in a first carbohydrate. In one embodiment, said first carbohydrate is not glucose.

In a second aspect, a method is provided for biomass production of yeast cells, comprising the step of stimulating the respiration in said yeast cells. In the presence of glucose in the growth medium yeast cells spontaneously start fermenting said glucose and simultaneously repress the metabolism of alternative carbon sources as well as respiration. “Biomass production of yeast cells” or “yeast biomass production” as used herein refers to the propagation of yeast cells excluding the process of fermentation. A whole industry is built on the commercial offering of batches of yeast as a starting culture for industrial fermentations. Although the production of yeast-derived molecules such as ethanol is to be avoided during the process of yeast biomass production, most often glucose is used a cheap carbon source for yeast growth. Yet considerable efforts are to be made in industrial settings to prevent fermentation in the presence of glucose, including intensive aeration, intensive mixing and carefully dosing glucose. This implies a close monitoring of the process, extra costs and still an uncertain outcome. The current application solves these technical problems by providing means and methods for biomass production of yeast cells in the presence of glucose, comprising the step of stimulating the respiration in said yeast cells. By said teaching biomass production of yeast is improved and made economically more efficient.

In a third aspect, a method is provided for the production of wine or beer with reduced alcohol levels, comprising providing yeast cells for the production of said wine or beer and stimulating respiration in said yeast cells before or during the wine or beer production process. From both a societal and consumer point of view there is an increasing demand for alcoholic beverages with reduced levels of alcohol, more particularly ethanol. Because of global warming and the resulting higher sugar concentrations in grapes, increasing levels of ethanol in wine are a growing concern of wine producers. Wines with levels of 15% alcohol by volume or more is generally perceived as disturbing because of the negative impact on taste experience and food pairing and the potential risk of alcohol intoxication. Aborting fermentation processes when desired ethanol levels are reached is not a solution in the wine industry as the residual sugar fraction will turn dry wines into sweet wines. Also in the beer industry, efforts are made to produce alcohol-free beer or beer with limited amounts of alcohol. Current solutions include filtering out the ethanol from the finished beer or adding an ethanol-consuming respiration process. It goes without saying that this adds complexity to the process and implies the use of and thus investments in special apparatuses.

The current application solves these technical problems by providing means and methods for stimulating respiration during the production of wine or beer. Chemicals, biologicals, mutant yeasts or genetically adapted yeast cells that enhance respiration in the yeast culture will reduce the amount of glucose that will be used for ethanol production, increase the respiration::fermentation ratio and hence lead to beverages with reduced alcohol levels.

Alcohol by volume (abbreviated as ABV, abv, or alc/vol) is a standard measure of how much alcohol (i.e. ethanol) is contained in a given volume of an alcoholic beverage (expressed as a volume percent or vol %). It is defined as the number of milliliters (ml) of pure ethanol present in 100 ml of solution at 20° C. The number of milliliters of pure ethanol is the mass of the ethanol divided by its density at 20° C., which is 0.78924 g/ml. The ABV standard is used worldwide. In particular embodiments of the third aspect, the methods for the production of wine or beer with reduced alcohol levels are provided, wherein said reduced alcohol levels are at least 10%, at least 20%, at least 30%, at least 40%, at least 50%, at least 60%, at least 75%, at least 90% or 100% less compared to alcohol levels reached without stimulation of respiration. In other particular embodiments, the methods described herein for the production of wine with reduced alcohol levels are provided wherein the reduced alcohol level is less than 12 vol %, less than 11 vol %, less than 10 vol %, less than 9 vol %, less than 8 vol %, less than 7 vol %, less than 6 vol %, less than 5 vol %, less than 4 vol %, less than 3 vol %, less than 2 vol %, less than 1 vol % or less than 0.5 vol %.

In other particular embodiments, the methods described herein for the production of beer with reduced alcohol levels are provided wherein the reduced alcohol level is less than 7 vol %, 6 vol %, 5 vol %, less than 4 vol %, less than 3 vol %, less than 2 vol %, less than 1 vol % or less than 0.5 vol %.

Oxidative respiration (herein used as alternative for respiration or cellular respiration) entirely relies on an active electron transport chain in the inner membranes of mitochondria and comprises a series of complexes that transfer electrons from electron donors through a series of electron acceptors to molecular oxygen as final acceptor of electrons. The mitochondrial electron transfer chain in Saccharomyces cerevisiae consists of Complex II and the two proton pumping units Complex III (i.e. the ubiquinol-cytochrome c reductase or bc1 complex) and Complex IV (i.e. the cytochrome c oxidase or CcO complex).

In current application it is demonstrated that the positive effect of respiratory activity in reducing the lag phase of a yeast culture depends on complexes III and IV of the electron transport chain. Hence, in specific embodiments of the first, second and third aspect of current application and their accompanying embodiments, stimulation of respiration refers to an enhanced activity of the electron transport chain, particularly of the complexes III and IV of said electron transport chain. Complexes III and IV both found in the inner membrane of mitochondria assemble into supercomplex structures which enhance the efficiency of electron transfer between complexes and cellular respiration. The two supercomplexes seen in yeast consist of a dimeric bc1 complex associated with one (trimeric supercomplex) or two (tetrameric supercomplex) CcO monomeric complexes on either side of bc1 (Cruciat et al 2000 J Biol Chem 275, 18093-18098; Schagger and Pfeiffer 2000 EMBO J 19, 1777-1783). Specific embodiments of the first, second and third aspect of current application and their accompanying embodiments are provided, wherein the stimulation of respiration refers to an enhanced activity and/or stability of the mitochondrial supercomplexes, more particular of the electron transport chain supercomplexes comprising complexes III and IV or alternatively phrased of the bc1-Cco supercomplexes.

Complex III or the bc1 complex comprises three subunits involved in electron transfer and proton pumping and seven to eight supernumerary subunits (Xia et al 1997 Science 277:60-66; Iwata et al 1998 Science 281:64-7115). The essential catalytic core consists of cytochrome b (Cob), cytochrome c1 (Cyt1), and the Rieske ironsulfur (FeS) protein Rip1. The assembly of bc1 in yeast proceeds in a modular pathway, with the mitochondrially encoded Cob seeding the assembly process (Zara et al 2007 FEBS J 274:4526-4539; Zara et al 2009 FEBS J 276:1900-1914). A series of early Cob containing core assembly intermediates exists, containing bc1 subunits Qcr7 and Qcr8 and assembly factors (Zara et al 2007 FEBS J 274:4526-4539; Crivellone et al 1988 J Biol Chem 263:14323-14333; Gruschke et al 2012 J Cell Biol 199:137-150; Kronekova and Rödel 2005 Curr Genet 47:203-212; Gruschke et al 2011 J Cell Biol 193:1101-1114). Additional subunits (Cor1, Cor2, Cyt1, Qcr6, and Qcr9) are added to form the late core intermediate lacking only Rip1 and the peripheral subunit Qcr10 (Zara et al 2009 FEBS J 276:1900-1914; Crivellone et al 1988 J Biol Chem 263:14323-14333; Cruciat et al 1999 EMBO J 18:5226-5233). This nonfunctional late core intermediate exists loosely associated with CcO, and the addition of Rip1 and Qcr10 forms the active bc1 complex. Additional modulators of Rip1 function are described, e.g. the AAA-ATPase Bcs1 that regulates the insertion of Rip1 into the late core assembly (Wagener et al 2011 Mol Cell 44:191-202) and Mzm1 acting as a Rip1 chaperone stabilizing Rip1 during the matrix maturation step (Cui et al 2012 Mol Cell Biol 32:4400-4409). The importance of Rip1 was further demonstrated by Cui et al (2014 J Biol Chem 289:6133-6141) which showed that expression of a Rip1 truncation comprising the N-terminal 92 residues of Rip1 but lacking the C-terminal FeS-containing globular domain that interacts with Mzm1 enhances stabilization of the two bc1-CcO supercomplexes. Expression of said truncation (designated herein as N-Rip1) in yeast resulted in elevated CcO activity, oxygen consumption and cellular respiration. Document Cui et al (2014 J Biol Chem 289:6133-6141) is hereby incorporated as reference. In particular embodiments of the herein described methods said respiration is increased by expressing an activator of respiration. In one embodiment said activator of cellular respiration encodes a truncated Rip1 protein. In a particular embodiment said truncated Rip1 protein comprises the N-terminal 92 residues of Rip1. In another embodiment, said truncated Rip1 protein is a fragment of the Rip1 protein lacking the C-terminal FeS-containing globular domain. The amino acid sequence of said C-terminal domain is referred to as SEQ ID No. 9. In another embodiment, said truncated Rip1 protein is N-Rip1 as depicted by SEQ ID No. 8 or as a sequence with a sequence homology of 90%, 91%, 92%, 93%, 94%, 95%, 96%, 97%, 98% or 99% to SEQ ID No. 8.

Besides Rip1, a series of other factors, including Rcf1, Rcf2, and Aac2, have been reported to be important for stabilization of the supercomplexes (Dienhart et al 2008 Mol Biol Cell 19:3934-3943; Claypool et al 2008 J Cell Biol 182:937-950; Strogolova et al 2012 Mol Cell Biol 32:1363-1373; Chen et al 2012 Cell Metab 15:348-360; Vukotic et al 2012 Cell Metab 15:336-347).

It is anticipated herein that increased expression or activity of one of the above described components or stabilizers of the bc1 and/or Cco complexes, lead to enhanced cellular respiration. Therefore the methods disclosed in the first, second and third aspect of current application are provided wherein said stimulation of respiration is achieved by enhancing the expression or activity of an activator of respiration in said yeast cells. In one embodiment, said activator is selected from the list consisting of Rcf1, Rcf2, Aac2, Cob, Cyt1, Rip1, N-Rip1, Bcs1, Mzm1, Qcr6, Qcr7, Qcr8, Qcr9, Qcr10, Crd1, Cor1 and Cor2. In another embodiment, said activator is Rcf1, Rcf2, Aac2, Cob, Cyt1, Rip1, N-Rip1, Bcs1, Mzm1, Qcr6, Qcr7, Qcr8, Qcr9, Qcr10, Crd1, Cor1 or Cor2. In a particular embodiment, said activator is Rip1, N-Rip1 or Qcr10. The amino acid sequences of Rip1, N-Rip1 and Qcr10 are depicted in SEQ ID No. 7, SEQ ID No. 8 and SEQ ID No. 12 respectively. In a more particular embodiment, said activator is N-Rip1. The skilled person is aware of several molecular techniques that can be used to express gene constructs in yeast and/or to enhance the expression level of specific genes. Briefly, this can be done by traditional molecular cloning techniques where an endogenous or exogenous promoter is operably linked with the coding sequence or the full sequence of a particular gene. Alternatively, the more recent Crispr-Cas technology can be used to modulate (e.g. to increase) the expression level of endogenous genes that positively influence respiratory activity.

In current application it is demonstrated that overexpression of the HAP4 transcription factor in yeast reduces the lag phase of said yeast during a glucose to ethanol or a glucose to galactose shift (FIG. 4B). Therefore the methods disclosed in the first, second and third aspect of current application are provided wherein said stimulation of respiration is achieved by enhancing the expression or activity of HAP4 in said yeast cells. Hap4 as used herein refers to a subunit of the heme-activated, glucose-repressed Hap2p/3p/4p/5p CCAAT-binding complex. HAP4 is also known as YKL109W and has the SGD ID:S000001592.

The nucleic acid sequence of the HAP4 gene is depicted in SEQ ID No. 10 and encodes a protein of 554 amino acids as depicted in SEQ ID No. 11. In a particular embodiment, HAP4 refers to SEQ ID No. 10 or to a nucleic acid molecule with at least 90%, at least 91%, at least 92%, at least 93%, at least 94%, at least 95%, at least 96%, at least 97%, at least 98% or at least 99% homology to SEQ ID No. 10. In another particular embodiment, HAP4 refers to a nucleic acid molecule encoding SEQ ID No. 11 or an amino acid sequence with at least 90%, at least 91%, at least 92%, at least 93%, at least 94%, at least 95%, at least 96%, at least 97%, at least 98% or at least 99% homology to SEQ ID No. 11.

Increasing cellular respiration of a yeast culture as provided in the methods of current application can also be achieved by adding an agonist of yeast respiration to the growth medium. Indeed, also pharmacologically respiration and the underlying electron transport chain can be stimulated. In one embodiment said agonist of yeast respiration is a chemical compound, a small molecule or a biological. Tests to know whether a molecule is an agonist of respiration are straightforward to perform and are known to the person skilled in the art. One example (without the purpose of limiting) to determine the respiratory activity of a yeast culture is by measuring the oxygen consumption of said yeast culture (reference is made to Cui et al 2014 J Biol Chem 289:6133-6141).

In particular embodiments, said chemical compound, small molecule or biological is a stimulator of complexes III and IV of the electron transport chain, more particularly said stimulator increases the activity of said complexes and thus oxidative respiration. Increasing oxidative respiration or increasing the activity of the mitochondrial electron transport chain can also be obtained by adding a stabilizer of the bc1 or Cco complexes or of the bc1-Cco supercomplexes to the growth medium of yeast cells. It was for example shown that cardiolipin is essential in supporting the critical interaction between complexes III and IV which stabilizes the supercomplexes in vivo. Cardiolipin plays an essential role in supercomplex formation as well (Zhang et al 2002 J Biol Chem 277, 43553-43556; Zhang et al 2005 J Biol Chem 280:29403-29408). Cardiolipin is produced by cardiolipin synthase which is encoded by the CRD1 gene. Hence, it is herein disclosed that cellular respiration according to the methods herein described can be enhanced be adding cardiolipin to the growth medium. Alternatively, it is also envisaged to enhance cellular respiration by increasing the expression of cardiolipin synthase or Cdr1 in the yeast cell which are used for the methods disclosed in current application.

Reducing the Expression of YLR108C Reduces the Lag Phase

Additional to the teaching that expression of known components and regulators of the mitochondrial electron transport chain can be used to enhance cellular respiration in yeast and thus are useful in the methods disclosed herein, a bulk segregant QTL analysis was performed in a search for novel mutant alleles reducing the lag phase. FIG. 7 is a schematic representation of the crucial steps in said analysis. To construct the segregating pools, we first crossed and sporulated a long-lag strain DBVPG1106 (MATa hoΔ::URA3 ura3Δ::NAT) with a short-lag strain DBVPG6044 (MATα hoΔ::URA3 ura3Δ::HYG). We performed tetrad dissection and subsequently screened 402 isolated spores for their lag phase length for a gradual shift from glucose to ethanol. From the distribution of lag lengths, three pools (each containing 30 spores) were constructed: a “short lag pool” containing spores on the short lag tail of the distribution, a “long lag pool” containing spores on the long lag tail of the distribution, and a “random pool” containing spores from every part of the distribution. Subsequently, DNA was extracted from the parental strains and the segregating pools using the Qiagen Genomic Tip 100G and sequenced using Illumina NextSeq (coverage of 800×/pool). The raw reads were aligned against the reference genome of the short-lag strain and further analyzed using the GATK best practices variant discovery pipeline. QTL mapping and discovery was performed using Multipool (Edwards and Gifford 2012 BMC Bioinformatics 13:S8) and EXPLoRA (Duitama et al 2014 BMC Genomics 15:207).

A major QTL was identified on chromosome XII. For each gene within this region, standard gene deletions and reciprocal hemizygosity analysis was performed. Interestingly, when gene of unknown function YLR108C was deleted in the haploid long-lag parent strain (DBVPG1106), its lag phase length shortens towards the lag length of the haploid parental short-lag strain (DBVPG6044). The same could be achieved by replacing the YLR108C allele from the long-lag parent by the mutant YLR108C allele from the short-lag strain. Although knock-out yeasts of YLR108C have been described (e.g. Zhu et al 2016 J Cell Sci 129:135-144), YLR108C has not been connected yet with respiration or modulation of lag phase. YLR108C or SGD ID S000004098 has a paralog YDR132C (SGD ID S000002539) that arose from whole genome duplication. Interestingly, further experiments revealed that YLR108C is a repressor of respiration. Moreover, the expression of YLR108C is controlled by glucose, suggesting that YLR108C might control an important step in the process of glucose-repression. The YLR108C data disclosed herein adds further evidence of the importance of respiration in the control of lag times.

Based hereon, the invention is further defined in the following aspects and embodiments.

In a fourth aspect, methods are provided to increase cellular respiration in yeast cells, comprising reducing the expression of YLR108C or YDR132C in said yeast cells. The application thus also provides the methods according to the first, second and third aspect of current application and their accompanying embodiments, wherein said stimulation of respiration is achieved by reducing the expression of a repressor of respiration in said yeast cells. In one embodiment, said repressor of respiration is or encodes for YLR108C or YDR132C.

The nucleic acid sequence of the YLR108C allele is depicted in SEQ ID No. 1 and encodes a protein of 485 amino acids as depicted in SEQ ID No. 2. In a particular embodiment, said repressor of respiration refers to SEQ ID No. 1 or to a nucleic acid molecule with at least 90%, at least 91%, at least 92%, at least 93%, at least 94%, at least 95%, at least 96%, at least 97%, at least 98% or at least 99% homology to SEQ ID No. 1. In another particular embodiment, said repressor of respiration refers to a nucleic acid molecule encoding SEQ ID No. 2 or an amino acid sequence with at least 90%, at least 91%, at least 92%, at least 93%, at least 94%, at least 95%, at least 96%, at least 97%, at least 98% or at least 99% homology to SEQ ID No. 2.

The nucleic acid sequence of the YDR132C allele is depicted in SEQ ID No. 5 and encodes a protein of 495 amino acids as depicted in SEQ ID No. 6. In a particular embodiment, said repressor of respiration refers to SEQ ID No. 5 or to a nucleic acid molecule with at least 90%, at least 91%, at least 92%, at least 93%, at least 94%, at least 95%, at least 96%, at least 97%, at least 98% or at least 99% homology to SEQ ID No. 1. In another particular embodiment, said repressor of respiration refers to a nucleic acid molecule encoding SEQ ID No. 6 or an amino acid sequence with at least 90%, at least 91%, at least 92%, at least 93%, at least 94%, at least 95%, at least 96%, at least 97%, at least 98% or at least 99% homology to SEQ ID No. 6.

In one embodiment, said “reducing the expression” means reducing the expression with at least 10%, at least 20%, at least 30%, at least 40%, at least 50%, at least 60%, at least 70%, at least 80%, at least 90%, at least 95%, or at least 97% compared to the expression level of said repressor in a wild-type situation. In a particular embodiment, said “reducing the expression” means reducing the expression with at least 50% or at least 75% compared to wild-type.

The person skilled in the art is well aware about the several means and methods available to reduce the expression of a gene in yeast. In one example, the expression can be reduced by interrupting the gene (e.g., by the insertion of a selectable marker gene) or by making it inoperative (e.g., by “gene knockout”). Methods for gene knockout and multiple gene knockout are well known. See, e.g. Rothstein, 2004, “Targeting, Disruption, Replacement, and Allele Rescue: Integrative DNA Transformation in Yeast” In: Guthrie et al., Eds. Guide to Yeast Genetics and Molecular and Cell Biology, Part A, p. 281-301; Wach et al., 1994, “New heterologous modules for classical or PCR-based gene disruptions in Saccharomyces cerevisiae” Yeast 10:1793-1808. Methods for insertional mutagenesis are also well known. See, e.g., Amberg et al., eds., 2005, Methods in Yeast Genetics, p. 95-100; Fickers et al., 2003, “New disruption cassettes for rapid gene disruption and marker rescue in the yeast Yarrowia lipolytica” Journal of Microbiological Methods 55:727-737; Akada et al., 2006, “PCR-mediated seamless gene deletion and marker recycling in Saccharomyces cerevisiae” Yeast 23:399-405; Fonzi et al., 1993, “Isogenic strain construction and gene mapping in Candida albicans” Genetics 134:717-728. Other methods to disrupt a gene in a microorganism include the use of nucleases, such as zinc-finger nucleases (ZFNs), Transcription Activator-Like Effector Nucleases (TALENs), meganucleases but especially the CRISPR-Cas system. “Nucleases” as used herein are enzymes that cut nucleotide sequences. These nucleotide sequences can be DNA or RNA. If the nuclease cleaves DNA, the nuclease is also called a DNase. If the nuclease cuts RNA, the nuclease is also called an RNase. Upon cleavage of a DNA sequence by nuclease activity, the DNA repair system of the cell will be activated. Yet, in most cases the targeted DNA sequence will not be repaired as it originally was and small deletions, insertions or replacements of nucleic acids will occur, mostly resulting in a mutant DNA sequence. ZFN are artificial restriction enzymes generated by fusing a zinc finger DNA-binding domain to a DNA cleavage domain. Zinc finger domains can be engineered to target desired DNA sequences, which enables zinc finger nucleases to target a unique sequence within a complex genome. By taking advantage of endogenous DNA repair machinery, these reagents can be used to precisely alter the genomes of simple and higher organisms. Other technologies for genome customization that can be used to knock out genes are meganucleases and TAL effector nucleases (TALENs, Cellectis bioresearch). A TALEN® is composed of a TALE DNA binding domain for sequence-specific recognition fused to the catalytic domain of an endonuclease that introduces double strand breaks (DSB). The DNA binding domain of a TAL effector nuclease is capable of targeting with high precision a large recognition site (for instance 17 bp). Meganucleases are sequence-specific endonucleases, naturally occurring “DNA scissors”, originating from a variety of single celled organisms such as bacteria, yeast, algae and some plant organelles. Meganucleases have long recognition sites of between 12 and 30 base pairs. The recognition site of natural meganucleases can be modified in order to target native genomic DNA sequences (such as endogenous genes). Another more recent and very popular genome editing technology is the CRISPR-Cas system, which can be used to achieve RNA-guided genome engineering. CRISPR interference is a genetic technique which allows for sequence-specific control of gene expression in prokaryotic and eukaryotic cells. It is based on the bacterial immune system-derived CRISPR (clustered regularly interspaced palindromic repeats) pathway and has been modified to edit basically any genome. By delivering the Cas nuclease (in many cases Cas9) complexed with a synthetic guide RNA (gRNA) in a cell, the cell's genome can be cut at a desired location depending on the sequence of the gRNA, allowing existing genes to be removed and/or new one added and/or more subtly removing, replacing or inserting single nucleotides (e.g. DiCarlo et al 2013 Nucl Acids Res doi:10.1093/nar/gkt135; Sander & Joung 2014 Nat Biotech 32:347-355).

Therefore, in a fifth aspect, the application also provides a chimeric gene construct comprising a promoter active in yeast operably linked to at least one Crispr guide RNA targeting a YLR108C or YDR132C allele. In a particular embodiment, said at least one Crispr guide RNA is two Crispr guide RNA molecules. Optionally said chimeric gene construct further comprises a 3′ end region involved in transcription termination or polyadenylation. Also the use of said chimeric gene construct is provided to increase the cellular respiration in yeast, more particularly to reduce the lag phase of a yeast or yeast culture, to increase or optimize the biomass production or propagation of yeast, to produce wine or beer with a reduced level of alcohol or to produce alcohol-free wine or beer.

The person skilled in the art is familiar with selecting suitable Crispr guide RNA molecules against a specific target. Several online tools are freely available. Below, some non-limiting examples of Crispr guide RNA (gRNA) molecules are listed:

>gRNA towards end of the YLR108C gene
GTTAACGACTCCAAATGTAA
>gRNA in beginning of the YLR108C gene
TAATGCGTCCGCGAAAAGAC
>gRNA towards end of the YLR108C gene
GGTTTTATGAAATCCAGTAG

In case 2 gRNA molecules are used to knock-out the YLR108C gene completely, below gRNAs are a non-limiting example.

>Fcut gRNA
TTCATGGTTTGGTAAAAGCT
>Rcut gRNA
ATATTCCTTCGTCCCAGAGA

In a particular embodiment, said promoter is selected from the list comprising pTEF1 (Translation Elongation Factor 1); pTEF2; pHXT1 (Hexose Transporter 1); pHXT2; pHXT3; pHXT4; pTDH3 (Triose-phosphate Dehydrogenase) also known in the art as pGADPH (Glyceraldehyde-3-phosphate dehydrogenase) or pGDP or pGLD1 or pHSP35 or pHSP36 or pSSS2; pTDH2 also known in the art as pGLD2; pTDH1 also known in the art as pGLD3; pADH1 (Alcohol Dehydrogenase) also know in the art as pADC1; pADH2 also known in the art as pADR2; pADH3; pADH4 also known in the art as pZRG5 or pNRC465; pADH5; pADH6 also known in the art as pADHVI; pPGK1 (3-Phosphoglycerate Kinase); pGAL1 (Galactose metabolism); pGAL2; pGAL3; pGAL4; pGAL5 also known in the art as pPGM2 (Phosphoglucomutase); pGAL6 also known in the art as pLAP3 (Leucine Aminopeptidase) or pBLH1 or pYCP1; pGAL7; pGAL10; pGAL11 also known in the art as pMED15 or pRAR3 or pSDS4 or SPT13 or ABE1; pGAL80; pGAL81; pGAL83 also know in the art as pSPM1; pSIP2 (SNF1-interacting Protein) also know in the art as pSPM2; pMET (Methionine requiring); pPMA1 (Plasma Membrane ATPase) also known in the art as pKTI10; pPMA2; pPYK1 (Pyruvate Kinase) also known in the art as pCDC19; pPYK2; pENO1 (Enolase) also known in the art as pHSP48; pENO2; pPHO (Phosphate metabolism); pCUP1 (Cuprum); pCUP2 also known in the art as pACE1; pPET56 also known in the art as pMRM1 (Mitochondrial rRNA Methyltransferase); pNMT1 (N-Myristoyl Transferase) also known in the art as pCDC72; pGRE1 (Genes de Respuesta a Estres); pGRE2; GRE3; pSIP18 (Salt Induced Protein); pSV40 (Simian Vacuolating virus) and pCaMV (Cauliflower Mosaic Virus). These promoters are widely used in the art. The skilled person will have no difficulty identifying them in databases. For example, the skilled person will consult the Saccharomyces genome database website (http://www.yeastgenome.org/) or the Promoter Database of Saccharomyces cerevisiae (http://rulai.cshl.edu/SCPD/) for retrieving the yeast promoters' sequences.

The use of the above described chimeric gene constructs is provided to increase cellular respiration in yeast or in a yeast culture. In one embodiment, said increase of cellular respiration reduces the lag phase of a culture of said yeast cells, particularly the lag phase associated with the depletion of glucose in the growth medium. Hence, the use of said chimeric gene construct is provided to reduce the lag phase of a yeast or yeast culture, particularly when said lag phase is associated with the depletion of glucose in the growth medium. In another embodiment, said increase of cellular respiration improves, enhances or optimizes biomass production of said yeast cells. This equivalent as saying that said increase of cellular respiration improves, enhances or optimizes the propagation of said yeast cells. Hence, the use of said chimeric gene construct is provided to increase or optimize the biomass production or propagation of yeast. In yet another embodiment, said increase of cellular respiration enables the production of alcohol-free beer or wine, or beer or wine with reduced alcohol levels. Thus, also the use of said chimeric gene construct is provided to produce wine or beer with a reduced level of alcohol or to produce alcohol-free wine or beer.

In a sixth aspect, a yeast strain is provided in which the YLR108C or YDR132C allele has been disrupted or deleted by using nuclease technology, more particularly by means of the CRISPR-Cas technology. In a particular embodiment, a yeast strain is provided in which the expression of YLR108C or YDR132C is reduced compared to a wild-type yeast strain, wherein said yeast strain with reduced expression of YLR108C or YDR132C is not a YLR108C or YDR132C knock-out or wherein said expression is not 100% reduced. Particularly, said “reduced expression” means an expression level that is at least 10%, at least 20%, at least 30%, at least 40%, at least 50%, at least 60%, at least 70%, at least 80%, at least 90%, at least 95%, or at least 97% reduced compared to the expression level of YLR108C or YDR132C in a wild-type situation.

In one embodiment the use of a yeast strain with reduced expression of YLR108C or YDR132C compared to a wild-type yeast is provided to reduce the lag phase of a culture of said yeast. In a particular embodiment said lag phase is associated with the depletion of glucose in the growth medium. In another embodiment, the use of a yeast strain with reduced expression of YLR108C or YDR132C compared to a wild-type yeast is provided to enhance yeast biomass production. In yet another embodiment, the use of a yeast strain with reduced expression of YLR108C or YDR132C compared to a wild-type yeast is provided to produce beer or wine with reduced alcohol levels.

In a seventh aspect, a mutant YLR108C allele according to SEQ ID No. 3 is provided. Also the mutant YLR108C protein according to SEQ ID No. 4 is provided in current application as well as a yeast strain comprising said mutant YLR108C allele. Said mutant allele can be used to increase respiration in yeast. As described in current application, the endogenous YLR108C allele from a yeast strain in which respiration needs to be increased, can be replaced by said mutant YLR108C allele. Therefore, the use of said mutant YLR108C allele is provided in all aspects and embodiments of current application to reduce the lag phase of a yeast culture, to optimize or enhance biomass production of yeast and/or to produce wine or beer with reduced alcohol levels.

Terminology as used in describing the aspects of the invention is described in the following sections. All living cells, including yeast, need energy for cellular processes such as pumping molecules into or out of the cell or synthesizing needed molecules. Adenosine triphosphate (ATP) is a special molecule which provides energy in a form that cells can use for cellular processes. ATP can be produced by respiration or fermentation depending on the presence of oxygen (O₂).

Oxidative respiration is one of the key ways a cell produces ATP to fuel cellular activity in the presence of oxygen. Typically aerobic organisms respire pyruvate completely to CO₂ with O₂ as the terminal electron acceptor, thereby making maximal use of energy transformations for ATP production. Nutrients that are commonly used in cellular respiration include sugar, amino acids and fatty acids. In the field of biochemistry, oxidative respiration comprises a set of metabolic reactions and processes that take place in the cell's mitochondria by which biochemical energy from nutrients is converted into ATP together with the release of waste products. The reactions involved in respiration are catabolic reactions, which break large molecules into smaller ones, releasing energy in the process, as weak so-called “high-energy” bonds are replaced by stronger bonds in the products.

Under anaerobic conditions (i.e. in the absence of oxygen), many cells including yeast use a process called fermentation to make ATP from the breakdown of glucose. The term fermentation is thus narrowly defined as the extraction of energy from carbohydrates in the absence of oxygen. Importantly, the absence of oxygen is not a requirement. Saccharomyces yeast is facultative aerobic and controls the switch between fermentation and respiration in response to the external glucose level (see earlier, glucose repression). Fermentation is thus rather a non-oxygen-requiring process than a strict anaerobic process. In yeast, the anaerobic fermentation produces ethanol and carbon dioxide as waste products and is therefore also referred to as alcoholic or ethanol fermentation. Although fermentation produces energy fast, it has two disadvantages compared to aerobic respiration. Fermentation produces much less ATP than aerobic respiration, and fermentation produces a toxic by-product (in yeast this is alcohol).

Fermentation and oxidative respiration begin the same way, i.e. with glycolysis. In fermentation, however, the pyruvate made in glycolysis does not continue through oxidation and the citric acid cycle, and the electron transport chain does not run. Because the electron transport chain is not functional (due to the absence of oxygen or due to glucose-repression), the NADH made in glycolysis cannot drop its electrons off in said chain to turn back into NAD⁺. Instead during alcoholic fermentation, NADH donates its electrons to acetaldehyde, regenerating the electron carrier NAD⁺ and forming ethanol. Throughout current application, “respiration” and “cellular respiration” are interchangeably used and for the means and methods described herein is restricted to “oxidative respiration in yeast”.

### Other Definitions

A “chemical compound” as used herein refers to a substance made from two or more different elements that have been chemically joined. Examples of compounds include water (H₂O), which is made from the elements hydrogen and oxygen, and table salt (NaCl), which is made from the elements sodium and chloride. Chemical compounds have a unique and defined chemical structure held together in a defined spatial arrangement by chemical bonds.

A “small molecule” as used herein (as in the field of molecular biology and pharmacology) refers to a low molecular weight (<900 daltons) organic compound that may regulate a biological process. Most drugs are small molecules. Larger structures such as nucleic acids and proteins, and many polysaccharides are not small molecules, although their constituent monomers (ribo- or deoxyribonucleotides, amino acids, and monosaccharides, respectively) are considered small molecules. Small molecules can have a variety of biological functions or applications, serving as cell signaling molecules, drugs in medicine, pesticides in farming, and in many other roles for example by inhibiting a specific function of a protein or disrupt protein-protein interactions These compounds can be natural (such as secondary metabolites) or artificial (such as peptidomimetics).

“Biological” as used here refers to a substance that is made from a living organism or its products. A biological can be composed of sugars, proteins, or nucleic acids or complex combinations of these substances, or may be living entities such as cells and tissues. Biologics are isolated from a variety of natural sources—human, animal, or microorganism—and may be produced by biotechnology methods and other cutting-edge technologies. A non-limiting example of a biological is an antibody.

“Compound” means any chemical or biological compound, including simple or complex organic and inorganic molecules, peptides, peptido-mimetics (peptide-like molecules), proteins, antibodies, carbohydrates, nucleic acids or derivatives thereof, lipids or hormone analogs that are characterized by low molecular weights, derived synthetically or from natural resources. The term “compound” also comprises “test compounds” or “candidate compounds”, i.e. compounds that are not used as such in commercial settings but that can be used for lead optimization.

The term “endogenous” as used herein, refers to substances (e.g. genes or proteins) originating from within an organism, tissue, or cell. Analogously, “exogenous” is any material originated outside of an organism, tissue, or cell, but that is present (and typically can become active) in that organism, tissue, or cell.

A “promoter” is a DNA sequence comprising regulatory elements, which mediate the expression of a nucleic acid molecule. For expression, the nucleic acid molecule must be linked operably to or comprise a suitable promoter which expresses the gene at the right point in time and with the required spatial expression pattern. The term “operably linked” as used herein refers to a functional linkage between the promoter sequence and the gene of interest, such that the promoter sequence is able to initiate transcription of the gene of interest or for example of a Crispr guide RNA. A promoter that enables the initiation of gene transcription in a eukaryotic cell is referred to as being “active”. To identify a promoter which is active in a eukaryotic cell, the promoter can be operably linked to a reporter gene after which the expression level and pattern of the reporter gene can be assayed. Suitable well-known reporter genes include for example beta-glucuronidase, beta-galactosidase or any fluorescent or luminescent protein. The promoter activity is assayed by measuring the enzymatic activity of the beta-glucuronidase or beta-galactosidase. Alternatively, promoter strength may also be assayed by quantifying mRNA levels or by comparing mRNA levels of the nucleic acid, with mRNA levels of housekeeping genes such as 18S rRNA, using methods known in the art, such as Northern blotting with densitometric analysis of autoradiograms, quantitative real-time PCR or RT-PCR (Heid et al., 1996 Genome Methods 6: 986-994). A “chimeric gene” or “chimeric construct” is a recombinant nucleic acid sequence in which a promoter or regulatory nucleic acid sequence is operably linked to, or associated with, a nucleic acid sequence that codes for a mRNA (optionally further encoding an amino acid sequence), such that the regulatory nucleic acid sequence is able to regulate transcription or expression of the associated nucleic acid coding sequence. Importantly, in a chimeric gene construct as used herein the regulatory nucleic acid sequence of the chimeric gene is not operably linked to the associated nucleic acid sequence as found in nature. The term “a 3′ end region involved in transcription termination or polyadenylation” encompasses a control sequence which is a DNA sequence at the end of a transcriptional unit which signals 3′ processing or polyadenylation of a primary transcript and is involved in termination of transcription. The control sequence for transcription termination or terminator can be derived from a natural gene or from a variety of genes. For expression in yeast the terminator to be added may be derived from, for example, the TEF or CYC1 genes or alternatively from another yeast gene or less preferably from any other eukaryotic or viral gene.

The present invention is described with respect to particular embodiments and with reference to certain drawings but the invention is not limited thereto but only by the claims. Any reference signs in the claims shall not be construed as limiting the scope. The drawings described are only schematic and are non-limiting. In the drawings, the size of some of the elements may be exaggerated and not drawn on scale for illustrative purposes. Where the term “comprising” is used in the present description and claims, it does not exclude other elements or steps. Where an indefinite or definite article is used when referring to a singular noun e.g. “a” or “an”, “the”, this includes a plural of that noun unless something else is specifically stated. Furthermore, the terms first, second, third and the like in the description and in the claims, are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein.

The terms or definitions provided herein are solely to aid in the understanding of the invention. Unless specifically defined herein, all terms used herein have the same meaning as they would to one skilled in the art of the present invention. Practitioners are particularly directed to Michael R. Green and Joseph Sambrook, Molecular Cloning: A Laboratory Manual, 4th ed., Cold Spring Harbor Laboratory Press, Plainsview, N.Y. (2012); and Ausubel et al., Current Protocols in Molecular Biology (Supplement 47), John Wiley & Sons, New York (1999), for definitions and terms of the art. The definitions provided herein should not be construed to have a scope less than understood by a person of ordinary skill in the art. It is to be understood that although particular embodiments, specific configurations as well as materials and/or molecules, have been discussed herein for cells and methods according to the present invention, various changes or modifications in form and detail may be made without departing from the scope and spirit of this invention. The Examples described below are provided to better illustrate particular embodiments, and they should not be considered limiting the application. The application is limited only by the claims.

Additional to the above detailed description of the invention, the following experimental details further enable the skilled person to put all details of the invention into practice.

Population Growth Lag Time Measurements (Antimycin a Treatment and Glucose-to-Galactose Shift).

All population growth measurements were performed using Bioscreen C machines (Growth Curves USA). For the gradual shift experiments, cells were inoculated in a 96-well plate containing YP plus 5% glucose (high-glucose [HG]) medium and serially diluted for growth overnight at 30° C. shaking at 900 rpm. Cultures at an OD600 of 0.1 were transferred to a new plate and serially diluted in fresh HG medium for growth overnight. The next day, cultures at an OD600 of 0.1 were washed three times into YP plus 0.5% glucose (low glucose [LG]) plus the appropriate carbohydrate source, with or without 3 μg/ml of antimycin A, and diluted so that the initial cell concentration for the Bioscreen C measurements was 10⁵ cells/ml. OD600 measurements were taken every 15 min for 7 days or until the cultures reached stationary phase.

The same procedure was followed for the stationary growth experiments, replacing the HG medium from pregrowth and the final growth medium with 5% galactose. The lag times were obtained by analyzing the growth curves with a homemade R script. Since the initial OD600 values are under the detection limit, the first three measurements were averaged to determine the background OD600 signal for each well. Once the background signal was subtracted, the nonlinearity between the optical density and the cell density was corrected using the following formula derived from Warringer and Blomberg (2003 Yeast 20:53-67): OD_{600,cx}=OD_{600,me}+0.499(OD_{600,me})²+0.191 (OD_{600,me})³

where the cx subscript stands for corrected and the me subscript stands for measured.

The growth rate was then obtained by calculating the discrete derivative of In(OD_{600,me}) versus time. The minimum growth rate (minR) was identified. In the case of two exponential phases in the growth curve (i.e., conditions with a mix of carbon sources in the medium), the maximum growth rate for each phase (maxR1, maxR2) was detected. Once these parameters were determined, the lag time was calculated by the time difference between two time points: the start and the end. The start of the lag is defined by the time corresponding to the intersection point between an exponential line touching the growth curve at the point corresponding to the maxR1 and a horizontal line crossing the point corresponding to the minR. The end of the lag is defined by the time corresponding to the intersection point between an exponential line touching the growth curve at the point corresponding to the maxR2 and a horizontal line crossing the point corresponding to the minR.

Oxygen Consumption Rate Measurement and Analysis.

Cells were inoculated in a 96-well plate containing HG medium and serially diluted for growth overnight at 30° C. shaking at 900 rpm. The next day, cultures at an OD600 of 0.1 were transferred to a new plate and serially diluted in fresh HG medium for growth overnight. The next day, 50-μl portions of cultures at an OD600 of 0.1 were inoculated in 100 μl of HG medium in a 96-well plate which has a fluorescent oxygen sensor embedded at the bottom of each well (OxoPlates; PreSens Precision Sensing). To calibrate the oxygen levels, air-saturated water and air-saturated HG medium were used as the 100% saturated condition, while the 0% saturated condition was measured by totally filling a well with water containing 10% Na₂SO₃ and covering the well with plastic seal. The OxoPlate with 5 biological replicates of each culture and all the calibration solutions was incubated for 24 h at 30° C. in an automatic plate reader (Infinite M200 Pro; Tecan). Fluorescence intensity and OD600 were measured every 15 min for all wells. To analyze the oxygen consumption rate during growth using the OxoPlates, a customized R script was used. To plot the growth curve, the background OD600 signal was subtracted from the OD600 measurements. Oxygen levels (oxygen partial pressure in percent air saturation) were calculated per time point using the calibration solutions, and the ratio of indicator phosphorescence to reference fluorescence of the optical sensor in the OxoPlate was determined. To plot the oxygen consumption rate, we corrected for the optical density of the cells by calculating the derivative of the smoothed oxygen level curve, divided by the corresponding optical density at that specific time point.

Construction of HAP4-OE and N-Rip1 Strain.

The HAP4-OE and N-Rip1 strains were made using standard transformation protocol with the S. cerevisiae strain AN63 (New et al 2014 PLoS Biol 12:e1001764) (BY4742 SAL1⁺ MAL⁺ MATa) as the background strain. Expression of N-Rip1 and overexpression of the HAP4 gene were verified with specific primers.

Construction of YLR108C Strains

YLR108C deletion strains were obtained using the standard transformation protocol. A PCR product was obtained after amplifying the KAN marker from the plasmid pUG6 with specific primers. The diploid SLP×LLP strain was constructed by mating the haploid SLP and haploid LLP. The SLP×LLP strains where one of the two alleles is deleted were constructed by mating a haploid wild-type strain (SLP/LLP) with a haploid deletion strain (LLP YLR108CΔ/SLP YLR108CΔ). YLR108C overexpression strains were obtained using the standard transformation protocol. A PCR product was obtained from the plasmid pYM-N15. LLP YLR108C-SLP (allele swapped strain) was obtained by first deleting YLR108C using a PCR product obtained from plasmid pSR101, and then introducing the SLP allele of YLR108C (PCR product from genomic DNA of SLP).

Gradual Shift Glucose-to-Ethanol and Glucose-to-Maltose

The cultures were pregrown for 2 overnights (30° C. shaking at 900 rpm) in 150 μl YPD5 (10 g/l yeast extract, 20 g/l peptone, 5% glucose) in 96-well plates. Cell density of the cultures was controlled by growing them in serial dilutions of 50-100 μl into 150 μl made across the plate. Incubation lasts maximally 24 hours, after which cultures are transferred to fresh medium. At each transfer, optical density (OD600) was measured using a plate reader, and only cultures with an OD600 smaller than 0.05 were selected to be transferred. After the second overnight, cultures with OD600<0.05 were washed 3 times into either YPD0.5 (10 g/l yeast extract, 20 g/l peptone, 0.5% glucose) for the glucose-to-ethanol shift, or YPD0.5+MAL5 (10 g/l yeast extract, 20 g/l peptone, 0.5% glucose, 5% maltose) for the glucose-to-maltose shift. Then, the cultures were diluted to an initial cell concentration of 105 cells/ml and transferred to the Bioscreen C plate reader to measure population density every 15 minutes for up to 7 days until the cultures reached stationary phase. The growth curves were analyzed, and lag times were extracted, using a homemade R script.

### EXAMPLES

### Example 1. Respiration as Modulator of the Lag Phase

As described above, a Bar-Seq screen surprisingly revealed oxidative respiration as an important determinant in the modulation of the lag phase. To confirm the Bar-Seq results 41 de novo deletion mutants (AN63 background) were constructed, each with one selected respiratory gene deleted. These selected genes include 35 subunits and assembly factors of the electron transport chain as well as several subunits of the heme activator protein (Hap) complex, the coenzyme Q, and the cytochrome c complexes. Next, the lag time of each of these mutants was examined in separate cultures when growing in mixed sugar environments (0.5% glucose plus 5% galactose) as well as their growth rate in stable sugar conditions (growing only in galactose). We found that the deletion of the investigated subunits generally resulted in a significantly longer lag time compared to the wild type, with cytochrome c, coenzyme Q, and the complexes III and IV of the electron transport chain being the ones affecting the lag time the most (FIG. 2A). The time to resume growth for a mutant in which a subunit of the above-mentioned complexes is deleted was about 2-fold longer than for the wild type (FIG. 2A). These results suggest that respiration and particularly the functionality of cytochrome c and complexes III and IV of the electron transport chain are critical for the cells to adapt to a new sugar in the environment.

To verify that these long lag times are indeed due to defects in respiration, we interfered on a pharmacological level by adding antimycin A to the growth medium. This compound binds to cytochrome c reductase, thereby inhibiting the electron flow through the electron transport chain and thus oxidative respiration (Alexander et al 1990 Enzyme Microb Technol 12:2-19). Addition of antimycin A prolonged the lag times up to 2.2-fold (FIG. 2B). Moreover, the addition of antimycin A to the media equalized the lag times of all mutants and the wild type, with the exception of the deletion mutants from complex V of the electron transport chain; their lag times became longer but not as long as for the wild type. Aside from the deletions of the subunits in complex V, the lag times of the deletion mutants are similar to the longest lag measured in a mutant without antimycin A added to the medium, suggesting that the changes in lag time in these mutants are indeed due to impaired respiration.

Second, the growth rate of each of the mutants was measured in stable sugar conditions (5% galactose; FIG. 2C). Deletion of 27 out of 41 respiration genes led to a significantly (P<0.05) lower growth rate compared to the wild type. In order to confirm that these deletion mutants have altered growth rates because of impaired respiration, we also measured growth rates in the presence of antimycin A (FIG. 2D). All mutants grow at equally low growth rates in the presence of antimycin A, lower than the one that is observed for the slowest growing mutants in the absence of this compound. This indicates that the decreased growth rates shown by many respiratory deletion mutants are due to a lack of respiration and thus that cells rely at least partly on respiration to metabolize galactose.

As a final confirmation, the switch from glucose to galactose was checked in the absence of oxygen. This is based on the unique property of S. cerevisiae to rapidly proliferate in the absence of oxygen with minimal supplements (Visser et al 1990 Appl Environ Microbiol 56:3785-3792). Oxygen deprivation enables yeast to tune the energy supply, as it disables cells from generating ATP via oxidative phosphorylation despite the presence of a fully functional respiratory chain. In line with the results above, cultivation in the absence of oxygen resulted in extremely long lag phases and long galactose consumption phases (FIGS. 3A-3B).

Finally, to demonstrate that the respiration-lag phase correlation is not restricted to the AN63 background, the observations were verified in CEN.PK background which is intensively used for fundamental and applied biotechnology. Also in this strain background, similar to the AN63 strain, both deletion of Rip1 (a complex III subunit) in aerobic conditions or growing wild type in anaerobic conditions led to a prolonged lag phase during the diauxic shift from glucose to galactose (FIG. 3C).

In summary, our results show that impaired respiration leads to a reduced galactose growth rate and longer lag times during shifts to galactose.

### Example 2. Overexpression of HAP4, a Global Regulator of Respiratory Genes, Shortens the Lag Time

The previous example clearly demonstrates that reduced respiratory activity lengthens lag phases. To investigate whether increased respiratory activity would lead to shortened lag phases, and hence would provide solutions to the significant problems in industrial fermentation settings as discussed above, the transcription factor HAP4 was overexpressed in S. cerevisiae. Hap4 is the main transcription factor controlling the expression of respiration genes in yeast (Blom et al 2000 Appl Environ Microbiol 66:1970-1973). First it was confirmed that the HAP4 overexpression strain (HAP4-OE) had a higher respiratory activity than the wild type by measuring the oxygen consumption rate and the growth of the yeast (FIG. 4A). This experiment was performed by growing the cells in glucose as the sole carbon source, a condition in which the cells should be primarily fermenting, and thus, little oxygen should be consumed. Next, we determined the lag time of HAP4-OE and wild type yeasts by performing population growth analysis during a glucose-to-galactose shift and as a control, also during a glucose-to-ethanol shift. Fully in line with the results from Example 1, it can be observed that during a gradual shift from glucose to ethanol, the overexpression of HAP4 causes a 40% reduction of the lag phase compared to wild-type cells (FIG. 4B). Interestingly, during the shift from glucose to galactose, the effect of HAP4 overexpression on the duration of the lag phase is even more pronounced, reducing the lag time to about half of that of wild-type cells (FIG. 4B). These results not only disclose that increased respiratory activity leads to reduced lag phases in yeasts but that the shortened lag phase does not depend on the nutrient to which the yeast has to switch after growing on glucose.

### Example 3. Expression of Respiratory Genes is Predictive of a Short Lag Time

The findings disclosed herein not only allow to modulate the lag phase but can also be used to develop a method to select yeasts with a short lag time. To add further evidence for the latter, we set out to assess the exact timing of the induction of the genes involved in respiration. Specifically, we measured the expression level of different representative subunits of each complex in the electron transport chain and of Gall, a common reporter for GAL gene expression (Venturelli et al 2015 PLoS Biol 13:1-24; Escalante-Chong et al 2015 PNAS 112:1636-1641; Acar et al 2010 Science 329:1656-1660). These proteins were tagged using a fluorescent reporter, and the expression within single cells was tracked using fluorescence time-lapse microscopy. The results are represented in so-called kymographs, where each horizontal line represents a tracked cell and the color indicates the mean fluorescence in this cell as time progresses. In order to see the time of induction, the mean fluorescence level was normalized to the initial level, and the color scale was adjusted to visualize small changes from the initial level. The moment when a particular cell started growing is indicated by a black square (FIGS. 5A-5C). Not all the cells recover growth after the shift to galactose. However, interestingly, only the cells inducing respiratory genes or genes involved in galactose metabolism are able to escape the lag phase and resume growth. The induction of these genes precedes the escape from the lag, especially when taking into account the maturation time of the fluorescent reporter and its detection limit.

Next, we investigated whether natural variation in respiratory activity during growth on glucose might explain some of the variation in lag duration observed between genetically different strains of S. cerevisiae and thus whether respiratory activity during glucose growth could be predictive for the length of the lag phase when switching to alternative nutrient sources. For this analysis, we used publicly available proteomics and transcriptomics data of 18 different natural S. cerevisiae strains grown in chemostats in glucose-rich conditions (Skelly et al 2013 Genome Res 23:1496-1504). The lag time of said strains were measured when the strains were shifted from glucose to galactose. These results reveal considerable differences in the lag duration of the strains, with lag times differing from almost 0 to 2.5 h (FIG. 6A). We then calculated the Spearman correlation coefficient between the lag duration of the different S. cerevisiae strains and their reported protein quantities. When correlating all the proteins investigated by Skelly et al. (2013 Genome Res 23:1496-1504) with the lag times we measured, the Spearman coefficients follow a normal distribution, meaning that there is no general bias of the data toward positive or negative correlations (FIG. 6B). When we examined only the proteins related to respiration, as determined by Steinmetz et al. (2002 Nat Genet 31:400-404), the distributions of correlation coefficients already shows a slight bias for negative values, indicating that low levels of proteins involved in respiration are correlated with long lag durations. However, when we calculated the correlations between lag duration and the levels of proteins linked to the different electron transport chain complexes, the distribution of coefficients clearly shifted to the negative side of the correlation, meaning that high expression of these proteins is predictive of a short lag time (FIG. 6B). This is particularly true for complexes III and IV, whose correlation coefficients are at the lower side of the spectrum. These findings are in agreement with our results obtained by deleting 41 different respiration genes, which point to complexes III and IV of the electron transport chain as the components with more influence on the speed of growth adaptation (Example 2; FIGS. 2A-2D).

In summary, these data reveal that natural variation in the expression of respiratory proteins of the electron transport chain correlates with the length of the lag phase, making the strains with higher expression of these proteins more likely to adapt quickly to nutritional changes in the environment. As such the data provide the necessary information to develop a screening assay or a method for selecting yeasts with reduced lag phases. The application thus provides a method to select yeasts with a short lag phase when said yeasts are switched from glucose to another carbon source, said method comprising the steps of measuring the expression level in said yeasts of at least one gene involved in oxidative respiration, wherein a high expression level of at least one of said genes is indicative for said yeasts to have a short lag phase when switched from glucose to another carbon source, more particularly ethanol, maltose, galactose or glycerol. In particular embodiments, said at least one gene involved in oxidative respiration is selected from the list consisting of CYT1, QCR7, NDI1, SDH2, COX6, COX9, ATP4 and ATP5. In other embodiments, said expression level is defined as high when said expression level is at least 50% higher than the average expression level of 10 random genes of said yeast.

Interestingly, the fact that the correlation between the length of lag phase and respiration is demonstrated for two different S. cerevisiae strains (i.e. AN63 and CEN.PK) but also for 18 natural S. cerevisiae strains strongly supports that the disclosed invention can be used universally for all S. cerevisiae strains.

### Example 4. Reduced Expression of YLR108C or YDR132C Increases Respiratory Activity in Yeast

A bulk segregant QTL analysis using a short and long lag phase yeast (see detailed description) revealed the YLR108C gene. To further test that YLR108C is indeed a modulator of the lag phase, the allele was deleted in the short and long lag phase yeasts. Deleting the YLR108C allele in the long lag phase yeast (from here referred to as YLR108C-LLP) significantly reduced the lag time to such an extent that it became similar to that of the short lag phase yeast. Deleting the YLR108C allele in the short lag phase yeast (from here referred to as YLR108C-SLP) could reduce the lag time even more (FIG. 8-9). The same results could be obtained by crossing the short lag phase with the long lag phase yeast (FIG. 8), demonstrating that reducing the expression level of YLR108C is sufficient to reduce the lag phase.

Next, the YLR108C-LLP was swapped with the YLR108C-SLP in the long lag phase yeast. This experiment showed that replacing the YLR108C-LLP by the YLR108C-SLP allele was as efficient as deleting the YLR108C-LLP allele (FIG. 9). In line with the finding that YLR108C is a regulator of the lag phase, overexpression of the YLR108C-LLP allele increased the lag phase significantly both in the short and long lag phase yeast (FIG. 9). Moreover, the lag phase reducing effect of reducing the YLR108C expression is found in different yeast backgrounds (FIG. 14), indicating the YLR108C is a generic regulator of respiration in yeast.

To investigate whether YLR108C regulation is specific for the glucose-to-ethanol lag phase, the strains were also tested during a gradual shift in nutrients from glucose to maltose or to galactose. Interestingly the YLR108C-SLP could also reduce the glucose-to-maltose lag phase as well as the glucose-to-galactose lag phase in the long lag phase yeast (FIG. 10).

YLR108C is a protein of unknown function. Yet, the results disclosed below put YLR108C forward as a modulator of respiration, more particularly as component in the process of glucose-repression. First, yeasts were grown in changing nutrient conditions (glucose-to-ethanol) and the MinR_OD was calculated, i.e. the population density at which the cells enter the lag phase. FIG. 11 illustrates the MinR_OD for the long lag phase yeast (LLP), the LLP strain overexpressing YLR108C-LLP, LLP wherein the YLR108C gene was deleted, LLP wherein the YLR108C gene was swapped with that of the short lag phase yeast (SLP) and the SLP strain. The higher the MinR_OD, the more energy (and biomass) is produced during growth on the small amount of glucose available, and thus the more respiratory activity the cells had on glucose. Consequently, a short lag phase strain in general would have a higher MinR_OD than a long lagged one. Knocking-out the long lag phase allele (or introducing the short lag phase allele in the long lag phase strain), increased the MinR_OD compared to the wild-type long lag strain (FIG. 11). Overexpression of the long lag allele decreased the MinR_OD dramatically, indicating that this strain did not respire as much.

Second, oxygen consumption was measured using OxoPlates. The lower the population density is at 50% oxygen consumption, the more respiratory activity the cells have. As can be seen in FIG. 12 and fully in line with the data disclosed herein, this correlates well with the lag time (R²=0.9492). Short lag phase yeasts (MR1) and yeasts wherein the long lag phase allele was knocked-out (KO_SV15) all show higher oxygen consumption compared to the long lag phase strain SV15.

These data together with the observation that the expression of YLR108C is induced by glucose and repressed by ethanol (FIG. 13), suggests that YLR108C is a glucose-induced repressor of respiration. Hence, knocking-out or reducing the expression of YLR108C in yeast increases the respiratory activity of yeasts. This invention provides solutions for several problems in industrial yeast settings, for example preventing stuck fermentation because of increased lag phases, optimizing and simplifying yeast biomass production and/or enabling production of beer and wine with reduced level of alcohol.

1.-4. (canceled)

5. A method reduce of reducing the lag phase of a culture of yeast cells, the method comprising stimulating respiration in the yeast cells.

6. The method according to claim 5 wherein stimulating respiration comprises:
adding an agonist of respiration to the growth medium,
enhancing the expression of an activator of respiration in the yeast cells and/or reducing the expression of a repressor of respiration in the yeast cells.

7. The method according to claim 6, wherein the activator of respiration is the Hap4 transcription factor or the truncated N-Rip1 protein.

8. The method according to claim 6, wherein the repressor of respiration is YLR108C or YDR132C.

9. The method according to claim 6, wherein the agonist or the activator activates or stabilizes complexes III and IV of the electron transport chain in the yeast cells.

10. The method according to claim 9, wherein the agonist is cardiolipin and the activator is selected from the group consisting of Rcf1, Rcf2, Aac2, Cob, Cyt1, Rip1, Qcr6, Qcr7, Qcr8, Qcr9, Qcr10, Crd1, Cor1 and Cor2.

11. The method according to claim 5, wherein the lag phase is associated with the depletion of glucose in the growth medium.

12. A method of selecting a yeast with a short lag phase, the method comprising:
measuring the expression level in the yeast of at least one gene involved in oxidative respiration.

13. The method of claim 12, wherein at least one gene involved in oxidative respiration is selected from the group consisting of Rcf1, Rcf2, Aac2, Cob, Cyt1, Rip1, Qcr6, Qcr7, Qcr8, Qcr9, Qcr10, Crd1, Cor1 consisting of CYT1, QCR7, NDI1, SDH2, COX6, COX9, ATP4, and ATP5.

14. The method according to claim 12, wherein the lag phase is the lag phase associated with a switch from glucose to another carbon source.

15. The method according to claim 5, wherein the stimulation is performed by expressing in the yeast cells a chimeric gene construct comprising a promoter active in yeast operably linked to a Crispr guide RNA targeting a YLR108C or YDR132C allele.

16. A method of producing a fermentation product, the method comprising
adding a yeast selected to have a short lag phase to a fermentation medium so as to produce a fermentation product, wherein the produced fermentation product is characterized by a statistically significantly reduced level of alcohol compared to that of the fermentation product produced by a yeast not selected to have a short lag phase.

17. The method according to claim 16, wherein the yeast is an engineered yeast characterized by enhanced expression of an activator of respiration and/or by reduced expression of a repressor of respiration compared to a non-engineered yeast.

18. The method according to claim 17, wherein the activator of respiration is selected from the list consisting of Hap4, N-Rip1, Rcf1, Rcf2, Aac2, Cob, Cyt1, Rip1, Qcr6, Qcr7, Qcr8, Qcr9, Qcr10, Crd1, Cor1 and Cor2, and wherein the repressor of respiration is YLR108C or YDR132C.
